(19) United States
(12) Patent Application Publication
(10) Pub. No.: US 2013/0046028 A1
(43) Pub. Date: Feb. 21, 2013

(54) Cosmetic Raw Material

(71) Applicants: Brian P. Deeth, Midland, MI (US); Haruhiko Furukawa, Chiba-shi (JP); Tatsuo Souda, Ichihara-shi (JP); Seiki Tamura, Ichihara-shi (JP)

(72) Inventors: Brian P. Deeth, Midland, MI (US); Haruhiko Furukawa, Chiba-shi (JP); Tatsuo Souda, Ichihara-shi (JP); Seiki Tamura, Ichihara-shi (JP)

(73) Assignees: DOW CORNING TORAY CO., LTD., Tokyo, JP (JP); DOW CORNING CORPORATION, Midland, MI (US)

(21) Appl. No.: 13/643,571

(22) PCT Filed: Apr. 28, 2011
(86) PCT No.: PCT/US11/34263

§ 371 Date: Oct. 26, 2012

Related U.S. Application Data
(60) Provisional application No. 61/329,128, filed on Apr. 29, 2010.

(51) Int. Cl. A61K 8/92; A61Q 1/10; A61Q 5/00; A61Q 1/06; A61Q 1/00; A61Q 17/04

(52) U.S. Cl. 514/777; 514/772; 514/785; 514/786

(57) ABSTRACT

A cosmetic raw material comprising the following components (A), (B), and (C): (A) a specific polyether-modified organopolysiloxane; (B) a chain-form silicone oil that is a liquid at 25° C., does not contain a cyclic structure, and does not contain a resinous structure; and (C) an oil that is a liquid at 30° C. and that characteristically (c1) has at least one hydroxyl group in each molecule, (c2) has from 0 to 3, as the number of moles of addition, oxyethylene units in each molecule, (c3) has an HLB value in the range from 0.1 to 6.0, and (c4) has an average molecular weight in the range from 200 to 7000.

### TECHNICAL FIELD

The present invention relates to a cosmetic raw material comprising (A) a polyether-modified organopolysiloxane, (B) a chain-form silicone oil, and (C) a special liquid oil.

The present invention more particularly relates to a cosmetic raw material obtained by incorporating—in a mixture comprising (A) a polyether-modified organopolysiloxane and (B) a chain-form silicone oil, which are generally poorly compatible and provide a mixture that is prone to undergo separation—a component (C) that is a compatibilizer for components (A) and (B) and that is an oil that is a liquid at 30° C., has at least one hydroxyl group in the molecule, has from 0 to 3, as the number of moles of addition, oxyethylene units, has an HLB value in the range from 0.1 to 6.0, and has an average molecular weight of 200 to 7000. This cosmetic raw material has the advantages of having an excellent storage stability, being semi-transparent to transparent, and being easy to incorporate in cosmetics.

### BACKGROUND ART

A variety of oils, e.g., silicone oils, ester oils, hydrocarbon oils, and so forth, are incorporated in cosmetic products, and each is used or deployed in conformity with its particular characteristics. For example, silicone oils offer the advantages of a light tactile feel, an excellent spreadability, an excellent water repellency, and a high level of safety. While these oils are frequently used in combinations in cosmetic formulations in order to exploit their respective advantages while compensating for their shortcomings, the further incorporation of water increases the value of a cosmetic in terms of the tactile feel or in terms of skin care due to the supply of moisture. This increase in value also occurs due to an inhibition of the oily feel due to the effect of the water, which yields a fresher skin sensation than for the absence of water.

Emulsification using a surfactant is typically carried out in order to achieve a stable blend of an oil and water in a cosmetic. When the oil system includes a silicone oil, the use of an organic surfactant alone is unlikely to yield a stable emulsion and is prone to have a negative effect on the use sensation of the cosmetic, and this has resulted in active efforts to development a silicone-type surfactant that would be advantageous in terms of tactile feel.

Among the silicone-type surfactants, polyether group-containing organopolysiloxanes, which are also known as polyether-modified silicones, have long been widely used in the cosmetics sector as surfactants and emulsifying agents and as dispersion aids for powders. Reference is made to Patent Documents 1 to 5 for their use as surfactants, to Patent Documents 6 and 7 for their use as emulsifying agents, and to Patent Document 8 for their use as dispersion aids for powders. In particular, oleophilic polyether-modified silicones having an HLB of not more than 6 can be effectively used for W/Si- and W/(Si+O)-type emulsions and creams.

Polyether-modified silicones have generally been produced by the addition to an organohydrogensiloxane of a polyether that has a reactive unsaturated group. However, when a structure that will provide a low HLB has been sought, the compatibility between the unreacted polyether that is typically present in the modified silicone and the copolymer reaction product has in some instances been unsatisfactory and separation into two phases has occurred.

A compatibilizing art that solves this problem is given in Patent Document 9. Patent Document 9 reports that a mixture having the form of a transparent solution is obtained when a polydimethylsiloxane-polyoxyalkylene copolymer given by the structural formula MD₄₀₀D^{PE}₄M wherein PE=C₃H₆O(EO)₂₅(PO)₂₅COCH₃, i.e., a polyether-modified silicone, a cyclic polydimethylsiloxane, and water are mixed at a weight ratio of 5:41.4:0.9. The dilution of this polyether-modified silicone with the cyclic polydimethylsiloxane alone does improve the handling properties, but cannot solve the problems of a cloudy appearance or phase separation, and the incorporation of a small amount of water is thus the key to stabilization. Since cyclic polydimethylsiloxanes and water are ingredients that are very often used in and for cosmetic raw materials, the sale of polyether-modified silicones as emulsifying agents formulated as low-viscosity solutions that incorporate these ingredients has even been adopted by cosmetic manufacturers as a logical and valuable proposition.

However, the cyclic polydimethylsiloxanes have fallen out of favor in recent years and there have been increasingly active efforts to replace them with chain-form polydimethylsiloxanes, i.e., chain-form silicone oils. Viewed from the standpoints of the volatilization rate and tactile feel, the chain-form silicone oil nearest to decamethylcyclopentasiloxane (D5), which is a representative cyclic polydimethylsiloxane, is reported to be a dimethylpolysiloxane having a kinematic viscosity at 25° C. of 2 mm2/s whose main component is MD3M. However, there are differences in the compatibility with organic components between D5 and 2cst, and, because 2cst has an unsatisfactory compatibility with organic systems, it has not been possible to ensure formulation stability in conventional cosmetic product formulations with just the simple substitution of 2cst for D5.

In actuality, while a transparent and stable solution is obtained using D5 in the compatibilized formulation of Patent Document 9, replacement with 2cst produced the problems of a milky appearance and the occurrence of separation into two phases with elapsed time. In addition, with this compatibilized formulation, the polyether-modified silicone concentration is low at approximately 11-12%, and due to this the degree of freedom for adjusting or planning cosmetic formulations is low.
[Patent Document 1] JP 61-293903 A
[Patent Document 2] JP 61-293904 A
[Patent Document 3] JP 62-187406 A
[Patent Document 4] JP 62-215510 A
[Patent Document 5] JP 62-216635 A
[Patent Document 6] JP 61-090732 A
[Patent Document 7] JP 2002-179797 A
[Patent Document 8] JP 10-167946 A
[Patent Document 9] JP 63-022514 A

### SUMMARY OF INVENTION

### Technical Problems to be Solved

The present invention seeks to solve the problems identified above and to provide a cosmetic raw material that has a semi-transparent to transparent appearance and that improves the handling when blended and storage stability of cosmetic raw materials that contain a mixture of (A) a polyether-modified organopolysiloxane and (B) a chain-form silicone oil that is a liquid at 25° C. and does not contain a cyclic structure or a resinous structure, said (A) polyether-modified organopolysiloxane being prone to undergo phase separation due to a generally low compatibility, being prone as a cosmetic raw material to exhibit problems with handling when blended and with storage stability, and being problematic with regard to securing an excellent appearance.

A second problem for the present invention is to provide a cosmetic raw material that can improve the degree of freedom in cosmetic formulation due to the low concentration of the polyether-modified organopolysiloxane in a compatibilized formulation containing (A) a polyether-modified organopolysiloxane and that, even when the cosmetic raw material contains a high concentration of this polyether-modified organopolysiloxane, makes possible the design of stable compatibilized formulations that have good handling characteristics.

### Solution to Problems

The present inventors attained the present invention as a result of intensive investigations in order to achieve the previously indicated objects. That is, the objects of the present invention are achieved by a cosmetic raw material comprising:
(A) a polyether-modified organopolysiloxane that has a specific structure;
(B) a chain-form silicone oil that is a liquid at 25° C., does not contain a cyclic structure, and does not contain a resinous structure; and
(C) an oil that is a liquid at 30° C. and that satisfies the following conditions (c1) to (c4):
   (c1) at least one hydroxyl group is present in each molecule,
   (c2) from 0 to 3, as the number of moles of addition, oxyethylene units are present in each molecule,
   (c3) an HLB value in the range from 0.1 to 6.0, and
   (c4) an average molecular weight in the range from 200 to 7000.

The co-incorporation of this component (C) makes it possible to provide a cosmetic raw material that can achieve the previously indicated objects by stabilizing and readily compatibilizing components (A) and (B), which are generally poorly compatible and are prone to undergo mixture separation.

In addition, the previously indicated objects can be favorably achieved by the selection for component (A) of a polyether-modified organopolysiloxane that has a polyether modifying group with a specific structure, and/or a polyether-modified organopolysiloxane that has a specific silylalkyl group and a specific suitable HLB value.

The previously described objects can similarly be favorably achieved by the selection for component (B) of a methylpolysiloxane that has a kinematic viscosity at 25° C. of not more than 20 mm²/s.

The previously described objects can also be favorably achieved by the selection for component (C) of an oil that satisfies conditions (c1) to (c4), that is a liquid at 30° C., and that is at least one selection from (C-1) higher alcohols, (C-2) fatty acid esters, (C-3) ethers, and (C-4) silicones that have at least one hydroxyl group in the molecule. In particular, the present invention can favorably achieve the previously described objects when component (C) is a structure that contains a C₁₀-30 monovalent hydrocarbyl group as a hydrophobic moiety and more specifically when component (C) contains at least one structure selected from the isostearyl group, isostearate ester groups, the oleyl group, and oleate ester groups. The previously described objects can similarly be favorably achieved when component (C) has, as a hydrophilic moiety, a structure derived from a polyhydric alcohol selected from sorbitan, sucrose, glycerol, polyglycerol, propylene glycol, and polypropylene glycols. The previously indicated objects of the present invention can also be achieved when component (C) is at least one silicone selected from alcohol-modified silicones, silanol-modified silicones, and phenol-modified silicones and has a kinematic viscosity at 25° C. of not more than 200 mm²/s.

That is, the present invention is

“[1]A cosmetic raw material comprising the following components (A), (B), and (C):
(A) a polyether-modified organopolysiloxane that is represented by general formula (1) below;
(B) a chain-form silicone oil that is a liquid at 25° C., does not contain a cyclic structure, and does not contain a resinous structure; and
(C) an oil that is a liquid at 30° C. and that satisfies the following conditions (c1) to (c4):
   (c1) at least one hydroxyl group is present in each molecule,
   (c2) from 0 to 3, as the number of moles of addition, oxyethylene units are present in each molecule,
   (c3) an HLB value in the range from 0.1 to 6.0, and
   (c4) an average molecular weight in the range from 200 to 7000 general formula (1):

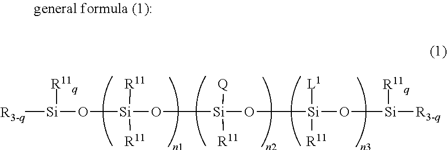

wherein
R¹¹ is a substituted or unsubstituted C₁₋₃₀ monovalent hydrocarbyl group, C₁₋₃₀ alkoxy group, the hydroxyl group, or the hydrogen atom,
L¹ is the siloxane dendron structure-containing silylalkyl group Lⁱ represented by the following general formula (2-1) when i=1, or is a chain-form organosiloxane group represented by the following general formula (2-2) or (2-3)

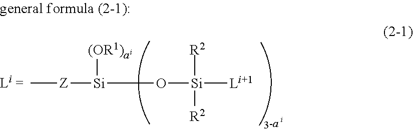

wherein each R¹ is independently a C₁₋₁₀ alkyl group or an aryl group, R² is a C₁₋₆alkyl group or the phenyl group, Z is a divalent organic group, i indicates the generation of the silylalkyl group represented by Lⁱ and is an integer from 1 to c, where c is the number of generations, which is the number of iterations of the indicated silylalkyl group, the number of generations c is an integer from 1 to 10, Lⁱ⁺¹ is the indicated silylalkyl group when i is less than c and is the methyl group or phenyl group when i=c, and aⁱ is a number in the range from 0 to 3

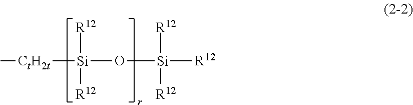

wherein R¹² is a C₁₋₃₀ monovalent hydrocarbyl group, the hydroxyl group, or the hydrogen atom, t is a number in the range from 2 to 10, and r is a number in the range from 1 to 100

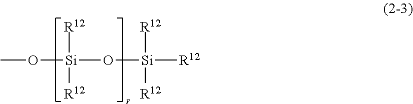

wherein R¹² is a group as defined above and r is a number in the range from 1 to 100,
Q is a polyether-modified group that characteristically is bonded to the silicon atom across an at least divalent linker group and contains the oxyalkylene unit represented by the following general formula (3-1) wherein at least 3 of the total oxyalkylene units are the oxyethylene unit general formula (3-1):

—CrH₂ᵣ—O— (3-1)
wherein r is a number in the range from 1 to 6,
   R is a group selected from L¹, Q, and R¹¹
   n1, n2, and n3 are numbers in the ranges 4≦n1≦1000, 0≦n2≦50, and 0≦n3≦50,
   q is an integer in the range from 0 to 3, and
   when n2=0, q is an integer in the range from 1 to 3 and at least one R is Q.
   [2] The cosmetic raw material according to claim 1, wherein component (A) is a polyether-modified organopolysiloxane characterized in that Q in general formula (1) is a polyether-modified group represented by the following general formula (4-1) general formula (4-1):

—R³(—O—X¹ₘ—R⁴)ₚ (4-1)

wherein R³ is a (p+1)-valent organic group, p is a number from 1 to 3, each X¹ is independently an oxyalkylene unit with the previously indicated general formula (3-1)

wherein at least three of the total oxyalkylene units represented by X¹ₘ are the oxyethylene unit, m is a number in the range from 3 to 100, and R⁴ is a group selected from the group consisting of the hydrogen atom, C₁₋₂₀ alkyl groups, and acyl groups.

[3] The cosmetic raw material according to [1] or [2], wherein component (A) is a polyether-modified organopolysiloxane characterized in that Q in general formula (1) is a polyether-modified group that contains a polyoxyalkylene unit represented by the following general formula (3-1-1)

general formula (3-1-1):

—(C₂H₄O)ₜ₁(C₃H₆O)ₜ₂— (3-1-1)

wherein t1 is a number greater than 3, t2 is a number greater than or equal to 0, and (t1+t2) is a number in the range from 4 to 100 and preferably is a number in the range from 8 to 50.
[4] The cosmetic raw material according to any one of [1] to [3], characterized in that component (A) is a polyether-modified organopolysiloxane characterized in that in general formula (1) L¹ is a silylalkyl group represented by general formula (2-1-1) or (2-1-2) below and when n3=0, q is an integer in the range from 1 to 3 and at least one of R is L¹

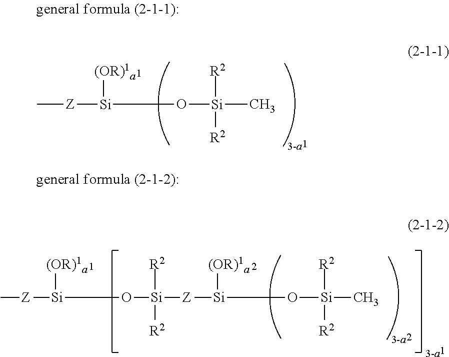

wherein R¹, R², and Z are groups as defined above and a¹ and a² are each independently a number in the range from 0 to 3.
[5] The cosmetic raw material according to any one of [1] to [4], characterized in that the HLB of component (A) is in the range from 0.1 to 6.0.
[6] The cosmetic raw material according to any one of [1] to [5], characterized in that component (B) is a chain-form silicone oil selected from (B-1) chain-form dimethylpolysiloxanes that have a kinematic viscosity at 25° C. of not more than 20 mm²/s or (B-2) chain-form alkyl-modified methylpolysiloxanes that have a kinematic viscosity at 25° C. of not more than 20 mm²/s.
[7] The cosmetic raw material according to any one of [1] to [6], characterized in that component (C) is a nonionic surfactant or a nonionic cosurfactant.
[8] The cosmetic raw material according to any one of [1] to [7], characterized in that component (C) is at least one oil selected from (C-1) higher alcohols, (C-2) fatty acid esters, (C-3) ethers, and (C-4) silicones that have at least one hydroxyl group in the molecule (excluding silicones that correspond to component (A) or component (B)).
[9] The cosmetic raw material according to [8], characterized in that component (C) is (C-1) a higher alcohol, (C-2) a fatty acid ester, or (C-3) an ether and has in its hydrophobic moiety a C₁₀₋₃₀ monovalent hydrocarbyl group.
[10] The cosmetic raw material according to [8] or [9], characterized in that component (C) is (C-2) a fatty acid ester or (C-3) an ether and is a derivative obtained by the esterification or etherification of a polyhydric alcohol selected from sorbitan, sucrose, glycerol, polyglycerols, propylene glycol, and polypropylene glycols.
[11] The cosmetic raw material according to [8], characterized in that component (C) is at least one silicone selected from alcohol-modified silicones, silanol-modified silicones, and phenol-modified silicones and has a kinematic viscosity at 25° C. of not more than 200 mm²/s.
[12] The cosmetic raw material according to any one of [1] to [11], that has the appearance at 25° C. of a semi-transparent to transparent liquid.”.

### Advantageous Effects of Invention

The present invention provides, in accordance with the first problem of the present invention as described above in “Problems to Be Solved by the Invention”, a cosmetic raw material that has a uniform, semi-transparent to transparent appearance and that improves the storage stability and handling properties upon blending of cosmetic raw materials that contain a mixture of (A) a polyether-modified organopolysiloxane and (B) a chain-form silicone oil that is a liquid at 25° C. and does not contain a cyclic structure or a resinous structure.

The present invention also provides, in accordance with the second problem of the present invention, a cosmetic raw material that can improve the degree of freedom in cosmetic formulation due to the low concentration of the polyether-modified organopolysiloxane in a compatibilized formulation containing (A) a polyether-modified organopolysiloxane and that, even when it contains a high concentration of this polyether-modified organopolysiloxane, can provide a stable compatibilized formulation that has good handling characteristics.

### BEST MODE FOR CARRYING OUT THE INVENTION

The present invention is a cosmetic raw material comprising the components (A), (B), and (C) indicated below, and the previously described effects of the invention can be achieved by the combination of these three components. These components are described in detail below.
(A) a polyether-modified organopolysiloxane that is represented by general formula (1) below
(B) a chain-form silicone oil that is a liquid at 25° C., does not contain a cyclic structure, and does not contain a resinous structure
(C) an oil that is a liquid at 30° C. and that satisfies the following conditions (c1) to (c4):
   (c1) at least one hydroxyl group is present in each molecule,
   (c2) from 0 to 3, as the number of moles of addition, oxyethylene units are present in each molecule,
   (c3) an HLB value in the range from 0.1 to 6.0, and
   (c4) an average molecular weight in the range from 200 to 7000.

Component (A)

The polyether-modified organopolysiloxane represented by general formula (1) is a straight-chain polyether-modified organopolysiloxane that has a degree of polymerization in a specific range; that has, in side chain position or terminal position in the molecule, a polyether-modified group that contains at least three oxyethylene units; and that may have, as a substituent in addition to the polyether-modified group, a siloxane dendron structure-containing silylalkyl group or a chain-form organosiloxane group.

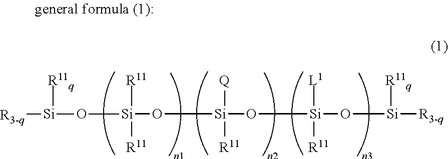

Each R¹¹ in general formula (1) is independently a substituted or unsubstituted monovalent C₁₋₃₀ hydrocarbyl group, a C₁₋₃₀ alkoxy group, the hydroxyl group, or the hydrogen atom. The substituted or unsubstituted monovalent C₁₋₃₀ hydrocarbyl group is a C₁₋₃₀ alkyl group, a C₆₋₃₀ aryl group, a C₆₋₃₀ aralkyl group, or a C₆₋₃₀ cycloalkyl group and can be exemplified by alkyl groups such as methyl, ethyl, propyl, butyl, pentyl, hexyl, heptyl, octyl, decyl, and so forth; cycloalkyl groups such as cyclopentyl, cyclohexyl, and so forth; and aryl groups such as phenyl, tolyl, and so forth. At least a portion of the hydrogen bonded to the carbon in these groups may be substituted by a halogen atom such as fluorine or by an organic group that contains, for example, an epoxy group, acyl group, carboxyl group, amino group, methacryl group, mercapto group, and so forth. The C₁₋₃₀ alkoxy group can be exemplified by lower alkoxy groups such as the methoxy group, ethoxy group, isopropoxy group, butoxy group, and so forth, and by higher alkoxy groups such as the lauryl alkoxy group, myristyl alkoxy group, palmityl alkoxy group, oleyl alkoxy group, stearyl alkoxy group, behenyl alkoxy group, and so forth. The methyl group, phenyl group, and hydroxyl group are particularly preferred for R¹¹. In another preferred embodiment, a portion of R¹¹ is the methyl group and a portion is a C₈₋₃₀ long-chain alkyl group.

L¹ in general formula (1) is an optional substituent for the component (A) polyether-modified organopolysiloxane, and each L¹ is independently a group selected from siloxane dendron structure-containing silylalkyl groups and chain-form organosiloxane groups.

The siloxane dendron structure-containing silylalkyl group L¹ is the group Lⁱ with the following general formula (2-1) when i=1.

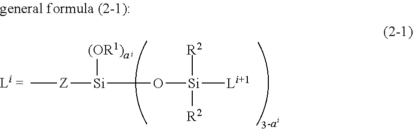

Each R¹ in the formula is independently a C₁₋₁₀ alkyl group or an aryl group and specifically is an alkyl group such as methyl, ethyl, propyl, butyl, pentyl, hexyl, heptyl, octyl, and so forth; a cycloalkyl group such as cyclopentyl, cyclohexyl, and so forth; or an aryl group such as phenyl and so forth.

R² is a C₁₋₆ alkyl group or is the phenyl group.

Z is a divalent organic group and can be specifically exemplified by the divalent organic group formed by the addition reaction to silicon-bonded hydrogen of a functional group that has an unsaturated hydrocarbyl group in terminal position, e.g., an alkenyl group or a carboxylate ester group such as the acryloxy group or methacryloxy group. However, there is no limitation to these functional groups, and the divalent organic group can be suitably selected in conformity with the method for introducing the silylalkyl group.

i indicates the generation of the silylalkyl group represented by Lⁱ and is an integer from 1 to c, where c is the number of generations, which is the number of iterations of the indicated silylalkyl group. The number of generations c is an integer from 1 to 10, and Lⁱ⁺¹ is the indicated silylalkyl group when i is less than c and is the methyl group or phenyl group when i=c. aⁱ is a number in the range from 0 to 3 and is preferably a number in the range from 0 to 2 and particularly preferably is 0 or 1.

L¹ is particularly suitably a silylalkyl group as represented by the following general formula (2-1-1) or general formula (2-1-2). R¹, R², and Z in these formulas are the previously defined groups and a¹ and a² are each independently numbers in the range from 0 to 3 and are each preferably in the range from 0 to 2 and particularly preferably 0 or 1.

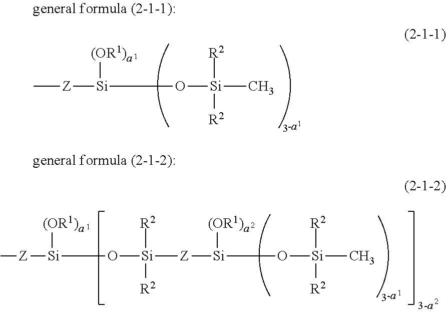

The siloxane dendron structure-containing silylalkyl group particularly preferably is represented by formula (2-1-1) and has a¹=0. Z is preferably a C₂₋₁₀ alkylene group as introduced by a reaction between silicon-bonded hydrogen and an alkenyl group or is preferably a divalent organic group as introduced by a reaction between silicon-bonded hydrogen and an unsaturated carboxylate ester group.

The chain-form organosiloxane group L¹ is a group represented by the following general formula (2-2) or (2-3).

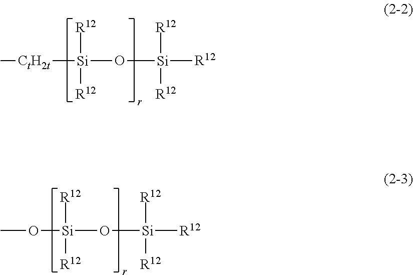

R¹² in general formulas (2-2) and (2-3) is a C₁₋₃₀ monovalent hydrocarbyl group, the hydroxyl group, or the hydrogen atom. The monovalent hydrocarbyl group can be exemplified by C₁₋₃₀ alkyl groups, C₆₋₃₀ aryl groups, C₆₋₃₀ aralkyl groups, C₆₋₃₀ cycloalkyl groups, and so forth. Specific examples of these are the same as previously described, while the methyl group, phenyl group, and hydroxyl group are particularly suitable.

hd t is a number in the range from 2 to 10 and r is a number in the range from 1 to 100 in general formulas (2-2) and (2-3). Since the chain-form organosiloxane group L¹ is hydrophobic, r is preferably a number in the range from 1 to 30, more preferably 1 to 20, and particularly preferably 2 to 10 from the standpoint of the balance with the hydrophilicity of the polyether functional group and the compatibility with the component (B) chain-form siloxane.

The component (A) polyether-modified organopolysiloxane must contain the special polyether-modified group described below, but preferably has in the molecule at least one functional group L¹ as described above and particularly preferably at least one silylalkyl group represented by the previously indicated general formula (2-1-1) or (2-1-2), because this can improve the compatibility with component (B) and can improve the compatibility for the obtained cosmetic raw material as a whole.

More specifically, it is particularly preferred for n3 in general formula (1) to be greater than or equal to 1, or, when n3=0, for q to be an integer in the range from 1 to 3 and for at least one of R to be L¹.

The Q in general formula (1) is an essential functional group for component (A) and is a polyether-modified group that characteristically is bonded to the silicon atom across an at least divalent linker group and contains the oxyalkylene unit represented by the following general formula (3-1) wherein at least 3 of the total oxyalkylene units are the oxyethylene unit

general formula (3-1):

—CrH₂ᵣ—O— (3-1)

wherein r is a number in the range from 1 to 6.

This Q can be specifically exemplified by the straight-chain or branched polyether-modified group represented by the following general formula (4-1) wherein p is an integer from 1 to 3.

general formula (4-1):

—R³(—O—X¹ₘ—R⁴)ₚ (4-1)

R³ in the preceding formula is a (p+1)-valent organic group and is the moiety that bonds the polyether-modified group Q to the silicon atom. There are no particular limitations on the structure of R³, but it can be exemplified by alkylene groups such as ethylene, propylene, butylene, hexylene, and so forth; alkylenephenylene groups such as ethylenephenylene, propylenephenylene, and so forth; alkylenearalkylene groups such as ethylenebenzylene; alkyleneoxyphenylene groups such as ethyleneoxyphenylene, propyleneoxyphenylene, and so forth; alkyleneoxybenzylene groups such as methyleneoxybenzylene, ethyleneoxybenzylene, propyleneoxybenzylene, and so forth; and the groups indicated below. R³ preferably has from 0 to 3 ether bonds and more preferably contains 0 or 1 ether bond.

Specific R³ groups that are trivalent or higher valent organic groups are given below.

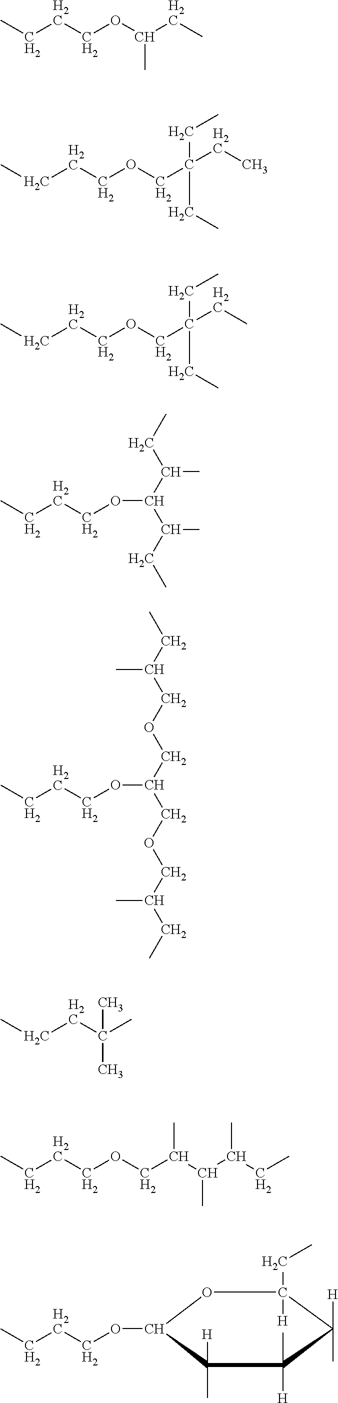

R³ is preferably a divalent organic group in the present invention, wherein alkylene is preferred among the previously described divalent organic groups and C₂₋₁₀ alkylene groups are particularly preferred.

Each X¹ is independently an oxyalkylene unit with the previously indicated general formula (3-1), and the structure shown by X¹ₘ is a polyoxyalkylene chain. It is necessary in the present invention for at least three of the oxyalkylene units constituting this polyoxyalkylene chain to be the oxyethylene unit. Thus, when m in general formula (4-1) is 3 and p is 1, all of the X¹'s are the oxyethylene unit. Viewed from the perspective of the hydrophilicity of the polyether-modified group, m, which indicates the number of oxyalkylene units bonded in the structure represented by —X¹ₘ—, is preferably a number in the range from 3 to 100, more preferably is a number in the range from 8 to 50, and particularly preferably is a number in the range from 12 to 45.

R⁴ is a group selected from the group consisting of the hydrogen atom, C₁₋₂₀ alkyl groups, and acyl groups.

The functional group Q in the present invention is particularly preferably a polyether-modified group that contains the polyoxyalkylene unit represented by the following general formula (3-1-1). The compatibility with the other components (B) and (C) is most favorably improved by the presence of this polyoxyalkylene chain structure composed of the polyoxyethylene unit and polyoxypropylene unit.

general formula (3-1-1):

—(C₂H₄O)ₜ₁(C₃H₆O)ₜ₂— (3-1-1)

In this formula, t1 is a number greater than 3, t2 is a number greater than or equal to 0, and (t1+t2) is a number in the range from 4 to 100 and preferably is a number in the range from 8 to 50. The absolute value of the difference between t1 and t2 is preferably not more than 20 and particularly preferably is not more than 10.

A favorable example of the functional group Q in the present invention is the straight-chain polyether-modified group represented by the following general formula (4-1-1).

—R^{3″}—O—(C₂H₄O)ₜ₁(C₃H₆O)ₜ₂—R⁴ (4-1-1)

R^{3′} in the preceding formula is a divalent organic group selected from the group consisting of alkylene groups, alkylenephenylene groups, alkylenearalkylene groups, alkyleneoxyphenylene groups, and alkyleneoxybenzylene groups, and C₂₋₁₀ alkylene groups are preferred. In addition, R⁴ is the same group as defined as above and t1 and t2 are the same numbers as defined above.

R in general formula (1) is a group selected from the previously described L¹, Q, and R¹¹.

n1, n2, and n3 are numbers in the ranges 4≦n1≦1000, 0≦n2≦50, and 0≦n3≦50 and q is an integer in the range from 0 to 3. When n2=0, q is an integer in the range from 1 to 3 and at least one R is Q. Thus, the polyorganosiloxane with general formula (1) necessarily has a polyether-modified group Q in side chain or terminal position.

Viewed from the perspective of the compatibility, n1, n2, and n3 are more preferably numbers in the ranges 4≦n1≦750, 2≦n2≦25, and 0≦n3≦25, but there is no limitation to these ranges.

Again viewed from the perspective of the compatibility, the HLB value of component (A) is similarly suitably in the range from 0.1 to 6 and is particularly suitably in the range from 0.2 to 4. This HLB value is determined by the following formula based on the molecular structure of component (A).

HLB value of Component (A)

HLB value=(sum of the mass % values for the oxyethylene units and OH groups in the molecule)/5

The polyether-modified organopolysiloxane with general formula (1) can be obtained by the addition reaction of a monounsaturated polyether compound that has a carbon-carbon double bond at one terminal of the molecular chain, to a polyorganosiloxane that has a reactive functional group and specifically an organopolysiloxane that has the silicon-hydrogen bond. There is no particular limitation on the type of addition reaction, but carrying out the addition reaction in the presence of a hydrosilylation reaction catalyst is preferred from the standpoints of the purity and yield and the ability to control the reaction.

Such polyether-modified silicones are known and are also commercially available.

The present invention further relates to a cosmetic raw material wherein the use is also preferred of a polyether-modified silicone that has been subjected to a known deodorization treatment, for example, a hydrogenation treatment, a hydrolysis treatment using an acidic substance, a process of stripping off the low-boiling fraction, and so forth.

Component (B)

Component (B) is a chain-form silicone oil that is a liquid at 25° C. and that does not contain a cyclic structure and does not contain a resinous structure. An object of the present invention is to provide a cosmetic raw material comprising a mixture of the previously described (A) polyether-modified organopolysiloxane and the (B) chain-form silicone oil that is a liquid at 25° C., does not contain a cyclic structure, and does not contain a resinous structure. This chain-form silicone oil is positioned as a component that is incorporated in a cosmetic as a replacement for cyclic silicone oils, e.g., decamethylcyclopentasiloxane (D5) and so forth.

This chain-form silicone oil, as long as it is a liquid at 25° C., may have a straight-chain structure or may have a chain-form structure that is branched due to the presence of 1 or 2 or more T units (SiO_{3/2}) and/or Q units (SiO_{4/2}) in each molecule. This chain-form silicone oil particularly preferably has a substantially straight-chain molecular structure.

The viscosity of this straight-chain silicone oil at 25° C. is generally in the range from 0.65 to 100,000 mm²/s, while the use of the range from 0.65 to 10,000 mm²/s is favorable.

The straight-chain silicone oil under consideration is suitably a straight-chain organopolysiloxane with the following structural formula.

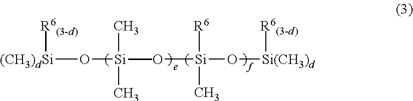

R⁶ in the preceding formula is the hydrogen atom or a group selected from C₁₋₃₀, monovalent unsubstituted or fluorine- or amino-substituted alkyl groups, aryl groups, and alkoxy groups. d is a number from 0 to 3 and e+f is a number in the range in which the viscosity of this straight-chain organopolysiloxane provides a liquid at 25° C.

The straight-chain organopolysiloxane can be more specifically exemplified by a dimethylpolysiloxane or organohydrogenpolysiloxane endblocked by the trimethylsiloxy group at both molecular chain terminals, methylphenylpolysiloxane endblocked by the trimethoxysiloxy group at both molecular chain terminals, dimethylsiloxane.methylphenylsiloxane copolymer endblocked by the trimethylsiloxy group at both molecular chain terminals, diphenylpolysiloxane endblocked by the trimethylsiloxy group at both molecular chain terminals, dimethylsiloxane diphenylsiloxane copolymer endblocked by the trimethylsiloxy group at both molecular chain terminals, trimethylpentaphenyltrisiloxane, phenyl(trimethylsiloxy)siloxane, methylalkylpolysiloxane endblocked by the trimethylsiloxy group at both molecular chain terminals, dimethylpolysiloxane methylalkylsiloxane copolymer endblocked by the trimethylsiloxy group at both molecular chain terminals, dimethylsiloxane.methyl(3,3,3-trifluoropropyl)siloxane copolymer endblocked by the trimethylsiloxy group at both molecular chain terminals, α,ω-diethoxypolydimethylsiloxane, 1,1,1,1,3,5,5,5-heptamethyl-3-octyltrisiloxane, 1,1,1,3,5,5,5-heptamethyl-3-dodecyltrisiloxane, 1,1,1,3,5,5,5-heptamethyl-3-hexadecyltrisiloxane, tristrimethylsiloxymethylsilane, tristrimethylsiloxyalkylsilane, tetrakistrimethylsiloxysilane, tetramethyl-1,3-dihydroxydisiloxane, octamethyl-1,7-dihydroxytetrasiloxane, hexamethyl-1,5-diethoxytrisiloxane, hexamethyldisiloxane, octamethyltrisiloxane, and so forth.

Viewed from the standpoint of use as a substitute for decamethylcyclopentasiloxane (referred to below simply as “D5”) in the cosmetic raw material, the viscosity of component (B) at 25° C. is preferably in the range from 0.65 to 100 mm²/s, particularly preferably in the range from 0.65 to 20 mm²/s, and most preferably in the range from 0.65 to 5 mm²/s. Component (B) may also be a mixture of two or more chain-form silicone oils having different viscosities.

A particularly favorable example from the standpoint of use as a substitute for D5 is a chain-form silicone oil selected from (B-1) chain-form dimethylpolysiloxanes that have a kinematic viscosity at 25° C. in the range from 0.65 to 20 mm²/s or (B-2) chain-form alkyl-modified methylpolysiloxanes that have a kinematic viscosity at 25° C. in the range from 0.65 to 20 mm²/s. Examples at a more specific level are a dimethylpolysiloxane endblocked by the trimethylsiloxy group at both molecular chain terminals and having a viscosity from 0.65 to 5 mm²/s and a methyltrisiloxane that has a C₈₋₂₀ alkyl group, e.g., 1,1,1,3,5,5,5-heptamethyl-3-octyltrisiloxane. These chain-form silicone oils are well suited for use as substitutes for cyclic silicone oils, e.g., D5 and so forth, and their use for component (B) of the present invention provides a cosmetic raw material that can form a stable compatibilized formulation with good handling characteristics.

Component (C)

Component (C) is an oil that is a liquid at 30° C. and that satisfies conditions (c1) to (C4), infra, and functions as a compatibilizer for the previously described components (A) and (B). In addition, the “liquid at 30° C.” means that component (C) of the present invention encompasses not only oils that are liquid at room temperature, i.e., 25° C., but also oils that do not exhibit fluidity but rather are a solid, semi-solid, or wax at room temperature, i.e., 25° C., and that when heated as whole to 30° C. exhibit a liquid state.
(c1) at least one hydroxyl group is present in each molecule,
(c2) from 0 to 3, as the number of moles of addition, oxyethylene units are present in each molecule,
(c3) an HLB value in the range from 0.1 to 6.0, and
(c4) an average molecular weight in the range from 200 to 7000

The first condition (c1) for component (C) is that at least one hydroxyl group is present in each molecule of component (C) wherein there are no particular limitations on the bonding regime for the hydroxyl group (—OH) in the molecule. In specific terms, the hydroxyl group may be bonded to a divalent hydrocarbyl group or may be bonded to a silicon atom as a silanol group or may be bonded to a carbonyl group —C(═O)— as in the carboxyl group —C(═O)OH.

From the standpoint of the compatibility-improving effect for the cosmetic raw material as a whole, the number of hydroxyl groups per molecule of component (C) is preferably in the range from 1 to 10 on average and the hydroxyl group is particularly preferably selected from the group consisting of alcoholic hydroxyl groups, phenolic hydroxyl groups, and the silanol group. The alcoholic hydroxyl group may be not only the alcoholic hydroxyl group present in a monohydric alcohol molecule such as isostearyl alcohol, but may also be the alcoholic hydroxyl group present in a polyhydric alcohol molecule, as represented by sorbitan, sucrose, glycerol, and polyglycerols, or present in the molecule of a derivative of the preceding.

The second condition (c2) for component (C) is that from 0 to 3, as the number of moles of addition, oxyethylene units are present in each molecule. This oxyethylene unit denotes —C₂H₄—O— where the —O— is an ether bond. For example, the number of moles of addition for the oxyethylene units in the substructure represented by —{C₂H₄—O}₂—CH₃ is 2. On the other hand, in the case of n-propyl alcohol, the —O— in CH₃—C₂H₄—O— is not an ether bond, but rather is part of an alcoholic hydroxyl group, and thus an oxyethylene unit is not present in the molecule and the number of moles of addition is 0.

From the standpoint of the compatibility-improving effect for the cosmetic raw material as a whole, the number of oxyethylene units per molecule of component (C), i.e., the number of moles of addition, is preferably from 0 to 2 and particularly preferably is 0 or 1 with 0 being the most preferred.

The third condition (c3) for component (C) is that its HLB value is in the range from 0.1 to 6.0. Excluding the case of the fatty acid esters of polyhydric alcohols, the HLB value is the value determined based on the average molecular structure of component (C) and is calculated from the following formula.

HLB value=(sum of the mass % values for the oxyethylene units and OH groups in the molecule)/5

When, on the other hand, component (C) is the fatty acid ester of a polyhydric alcohol, the value calculated as indicated below is used. In the formula below, S is the saponification value of the polyhydric alcohol ester and A is the acid value of the starting fatty acid.

HLB value=20×(1−S/A)

From the standpoint of the compatibility-improving effect for the cosmetic raw material as a whole, the HLB value of component (C) is preferably in the range from 0.2 to 5.5 and particularly preferably is in the range from 0.3 to 5.0.

The fourth condition (c4) for component (C) is that the average molecular weight must be in the range from 200 to 7000. The technical effect of improving the component (A)/component (B) compatibility cannot be manifested in the case of an oil with a molecular weight smaller than the lower limit for component (C), for example, when an oil is used that satisfies conditions (c1) through (c3) with regard to the features other than the molecular weight, such as dipropylene glycol (molecular weight=134). On the other hand, it may similarly not be possible to achieve a satisfactory improvement in the compatibility when the average molecular weight exceeds the previously indicated condition.

The average molecular weight is the value determined based on the average molecular structure of component (C). Accordingly, in the case of a polymer that presents a distribution in the degree of polymerization and molecular weight, the number-average molecular weight of the intended polymer is used. On the other hand, for an oil that has a defined average molecular structure, such as isostearyl alcohol, the average molecular weight used is the value calculated directly from the constituent atomic weights.

From the standpoint of the compatibility-improving effect for the cosmetic raw material as a whole, the average molecular weight of component (C) is preferably in the range from 220 to 3000, particularly preferably in the range from 230 to 1500, and most preferably in the range from 240 to 1000.

As the previously indicated HLB value and molecular weight show, component (C) is an oil that has surface activity and an average molecular weight of 200 to 7000 and also functions as a nonionic surfactant or cosurfactant. The use of this oil with the component (A) polyether-modified silicone can stabilize and readily compatibilize components (A) and (B), which are ordinarily poorly compatible and are prone to exhibit separation of their mixture. In addition, component (C) must have at least one hydroxyl group as a hydrophilic moiety, and, when it has a polyether moiety comprising more than 3 oxyethylene units as another hydrophilic moiety, it was found—as a result of the present investigations and with specific examples of the capacity for broadening or generalization—that this compatibility cannot be achieved.

Any component (C) of the present invention that is an oil that is a liquid at 30° C. and satisfies conditions (c1) to (c4) can be used without particular limitation; however, viewed from the perspective of the usefulness as a cosmetic raw material, component (C) can be favorably exemplified by at least one oil selected from (C-1) higher alcohols, (C-2) fatty acid esters, (C-3) ethers, and (C-4) silicones that have at least one hydroxyl group in the molecule, but excluding silicones that correspond to component (A) or component (B).

From the viewpoint of the compatibility-improving effect for the cosmetic raw material as a whole and in particular from the viewpoint of the improvement in storage stability in the case of long-term storage, component (C) preferably has a monovalent C₁₀₋₃₀ hydrocarbyl group as a hydrophobic moiety and particularly preferably has a C₁₂₋₂₀ alkyl group or alkenyl group as a hydrophobic moiety. This C₁₂₋₂₀ alkyl group or alkenyl group may be straight chain or branched chain and is favorably exemplified by C₁₋₂ alkyl (=lauryl), C₁₋₄ alkyl (=myristyl), C₁₋₆ alkyl (=palmityl), C₁₈ alkyl (=stearyl and isostearyl), and C₁₈ alkenyl (═oleyl) wherein the presence in the molecule of the isostearyl group or oleyl group is particularly preferred.

When component (C) is selected from the group consisting of (C-1) higher alcohols, (C-2) fatty acid esters, and (C-3) ethers, it particularly preferably has a C₁₂₋₂₀ alkyl or alkenyl group as described above and more preferably contains a C₁₈ alkyl or alkenyl group, e.g., the isostearyl group, isostearate ester group, oleyl group, oleate ester group, and so forth.

Suitable examples of component (C) when it is a (C-1) higher alcohol are C₁₂₋₂₀ alkyl alcohols and alkenyl alcohols. Viewed from the standpoint of being a liquid at 30° C., examples at a more specific level are lauryl alcohol, oleyl alcohol, isostearyl alcohol, hexyldodecanol, octyldodecanol, and so forth. Isostearyl alcohol is most preferred. Because these higher alcohols have an alcoholic hydroxyl group for the hydrophilic moiety and a C₁₂₋₂₀ alkyl or alkenyl group for the hydrophobic moiety, they exhibit a suitable surface activity and can as a result stabilize and readily compatibilize components (A) and (B).

When component (C) is (C-2) a fatty acid ester or (C-3) ether, component (C) is then preferably a derivative of a polyhydric alcohol selected from sorbitan, sucrose, glycerol, polyglycerols, propylene glycol, and polypropylene glycols. These component (C)'s can be obtained, for example, by the esterification with a fatty acid of a portion of the alcoholic hydroxyl groups of the corresponding polyhydric alcohol or by the etherification of a higher alcohol with a halide compound, and have another alcoholic hydroxyl group in the molecule. Favorable examples for the present invention are the sorbitan/fatty acid esters, glycerol/fatty acid esters, polyglycerol fatty acid esters, and propylene glycol/fatty acid esters that are the reaction products of a polyhydric alcohol as described above and a C₁₂₋₂₀ higher fatty acid. Similarly, favorable examples are also the alkyl glyceryl ethers and alkenyl glyceryl ethers that are the etherification products of a polyhydric alcohol as described above and a C₁₂₋₂₀ higher alcohol. Other favorable examples are the polyoxypropylene alkyl ethers and polyoxypropylene alkenyl ethers obtained by the addition of propylene oxide to a higher alcohol.

Examples at a more specific level when component (C) is a (C-2) fatty acid ester or (C-3) ether are glyceryl monoisostearate, glyceryl monooleate, polyglyceryl isostearate, polyglyceryl laurate, polyglyceryl myristate, polyoxypropylene stearyl ether, polyoxypropylene myristyl ether, polyoxypropylene lauryl ether, isostearyl glyceryl ether, oleyl glyceryl ether, sorbitan monooleate, sorbitan sesquioleate, sorbitan monoisostearate, sorbitan sesquiisostearate, and so forth. The presence of the isostearyl group is most favorable, e.g., glyceryl monoisostearate, propylene glycol monoisostearate, polyglyceryl isostearate, isostearyl glyceryl ether, sorbitan monoisostearate, and sorbitan sesquiisostearate.

Because these higher alcohols, fatty acid esters, and ethers have an alcoholic hydroxyl group for the hydrophilic moiety and a C₁₂₋₂₀ alkyl or alkenyl group for the hydrophobic moiety, they exhibit a suitable surface activity and in particular can as a result stabilize and readily compatibilize components (A) and (B). These higher alcohols, fatty acid esters, and ethers can be synthesized by known methods and in many cases are commercially available.

When component (C) is (C-4) a silicone that has at least one hydroxyl group in the molecule but excluding silicones that correspond to component (A) or component (B), component (C) is suitably, for example, at least one silicone selected from alcohol-modified silicones, silanol-modified silicones, and phenol-modified silicones, that has a kinematic viscosity at 25° C. of not more than 200 mm²/s.

A silicone having a relatively low degree of polymerization and a number-average molecular weight of 200 to 3000 and more favorably 200 to 1500 is particularly suitable for use as the silicone that has at least one hydroxyl group in the molecule. Viewed from the standpoint of the compatibility-improving effect for the cosmetic raw material as a whole, a favorable example is a silanol-modified silicone or phenol-modified silicone that has a kinematic viscosity at 25° C. of not more than 100 mm²/s. A specific example is an α,ω-hydroxypolydimethylsiloxane that has a kinematic viscosity at 25° C. of not more than 100 mm²/s.

The cosmetic raw material according to the present invention comprises the previously described components (A), (B), and (C), and the mixture of these components to homogeneity provides a cosmetic raw material that exhibits an excellent storage stability during storage at from low temperatures of 0° C. and below to high temperatures of 40° C. and above, and that exhibits an excellent compatibility among the individual components and an excellent stability when blended into a cosmetic. In addition, the cosmetic raw material according to the present invention is a stable liquid mixture that has a semi-transparent to transparent appearance at 25° C., assuming that a colored optional component is not admixed therein, and that does undergo separation or the production of sediment or precipitate.

In addition, as its second problem the present invention has as an object the introduction of a cosmetic raw material that enables the design of a good handling and stable compatibilized formulation—even when the compatibilized formulation containing component (A) contains a high concentration of component (A) in order to improve the degree of freedom in cosmetic formulation. Even when, in order to achieve this object, the cosmetic raw material according to the present invention is a compatibilized formulation that incorporates component (B) in the range from 10 to 200 mass parts and component (C) in the range from 5 to 100 mass parts per 100 mass parts component (A), the cosmetic raw material according to the present invention offers the advantages of an unimpaired storage stability and an unimpaired compatibility as described above.

In particular, the component (C) content can be selected as appropriate in conformity to the blending ratio between components (A) and (B) and is not particularly limited, but, taking as an example the use of component (A) and component (B) in a mass ratio of 4:6, the objects of the present invention can be particularly favorably achieved by incorporating component (C) at from 1 to 30 mass % and preferably at from 3 to 25 mass % of the cosmetic raw material as a whole.

Insofar as the objects of the present invention are not impaired, oily cosmetic raw materials other than the previously described components (A) to (C) may be blended as appropriate in the cosmetic raw material of the present invention. These optional oily cosmetic raw materials preferably have an excellent compatibility or can be mixed to uniformity with any of components (A) to (C).

These optional components can be exemplified by oil-soluble surfactants excluding those that correspond to component (A) or component (C), other oils excluding silicone oils that have a cyclic or resinous structure, the powders and/or colorants used in the usual cosmetics, silicone elastomers, oil-soluble gellants, silicone gums, ultraviolet protective components, acrylic silicone dendrimer copolymers, polyamide-modified silicones, alkyl-modified silicone waxes, alkyl-modified silicone resin waxes, organic resins, humectants, thickeners, preservatives, antibacterials, fragrances, salts, antioxidants, pH adjusters, chelating agents, algefacients, antiinflammatories, physiologically active components (whiteners, cell activators, agents for ameliorating skin roughness, circulation promoters, skin astringents, antiseborrheics, and so forth), vitamins, amino acids, nucleic acids, hormones, inclusion compounds, and so forth.

Since one object of the present invention is to improve the degree of freedom in cosmetic formulation, the quantity of incorporation of these optional components when designing a general-purpose cosmetic raw material is suitably less than 5 mass % of the cosmetic raw material as a whole, but there is no limitation to this.

On the other hand, in those instances in which the cosmetic that uses the present cosmetic raw material has been determined and/or the constituent components of this cosmetic have been determined and the preliminary incorporation of a prescribed quantity of a special oily cosmetic raw material is then required based on a consideration of blend stability or based on a consideration of simplifying the cosmetic production process, the incorporation of an oily cosmetic raw material other than the previously described components (A), (B), and (C) may then also make possible the design of a cosmetic raw material for specialty applications that may be particularized into individual cosmetics.

The cosmetic raw material of the present invention can be incorporated without particular limitation in those cosmetics that have heretofore used a cyclic polydimethylsiloxane and can also be incorporated with particular limitation in non-cosmetic topicals. As described in the preceding, the cosmetic raw material of the present invention provides an improved storage stability, an improved blend stability, and an improved degree of freedom in its formulation and offers the advantage of facilitating formulation design through its simple and direct substitution for conventional cyclic polydimethylsiloxanes.

Specific cosmetics can be exemplified by skin cosmetic products such as skin cleansing products, skin care products, make-up products, antiperspirant products, and UV protective products; by hair cosmetic products such as hair cleansing products, hair styling products, hair dyeing products, hair maintenance products, hair rinse products, hair conditioner products, and hair treatment products; and by bath cosmetic products. Similarly, the topicals can be exemplified by hair-restoring agents, hair-growth agents, analgesics, antiseptics, antiinflammatories, algefacients, and skin aging inhibitors, but are not limited to the preceding. Various forms can be selected for the products themselves, such as liquid, emulsion, solid, paste, gel, spray, and so forth.

### EXAMPLES

The description continues below with examples of the present invention, but the present invention is not limited by these examples. In the compositional formulas and structural formulas provided below, the methyl group is indicated by Me; the Me₃SiO or Me₃Si group is indicated by “M”; the Me₂SiO group is indicated by “D”; the MeHSiO group is indicated by “D^{H}”; and units provided by replacing the methyl in an M or D unit with any substituent (—R) are respectively indicated by M^{R} and D^{R}. In the production examples, examples, and tables provided below, dimethylpolysiloxane (2 mm²/s, 25° C.) denotes a dimethylpolysiloxane endblocked by the trimethylsiloxy group at both molecular chain terminals and having a kinematic viscosity at 25° C. of 2 mm²/s and having MD₃ M as its main component.

### Production Example 1

### Synthesis of Polyether-Modified Silicone No. 1

726.0 g of a methylhydrogenpolysiloxane with the average compositional formula MD₄₀₆D^{H}₄M, 212.6 g allyl polyether with the average structural formula CH₂═CH—CH₂—O(C₂H₄O)₁₉(C₃H₆O)₁₉H, 278 g isopropyl alcohol (IPA), and 1.88 g of a 1.5 weight % methanolic sodium acetate solution were introduced into a reactor and were heated to 60° C. while stirring under a nitrogen current. 3.25 g of a 1 weight % IPA solution of chloroplatinic acid was added and a reaction was run for 3 hours at 80° C. 2 g of the reaction solution was then recovered and the completion of the reaction was confirmed through gas production by alkali decomposition, wherein the residual Si—H group was decomposed with an aqueous ethanolic KOH solution and the conversion was calculated from the volume of hydrogen gas produced.

This reaction solution was diluted by the addition thereto of 0.37 g vitamin E and 1345 g dimethylpolysiloxane (2 mm²/s, 25° C.) with mixing. The low-boiling fraction other than the diluent was distilled out by heating the dilution under reduced pressure to obtain a mixture 1 comprising the chain-form dimethylpolysiloxane (2 mm²/s, 25° C.) and a composition containing a polyether group-containing organopolysiloxane with the average compositional formula MD₄₀₆D^{R}*²¹₄M; this polyether group-containing organopolysiloxane is referred to below as polyether-modified silicone No. 1 and had an HLB value of 1.8. The polyether-modified silicone:diluent ratio in mixture 1 was 40:60.

R*²¹ in the preceding formula indicates —C₃H₆O(C₂H₄O)₁₉(C₃H₆O)₁₉H. Mixture 1 was a uniform, whitish-brown viscous liquid immediately after production, but after two months at room temperature had undergone phase separation into a viscous grey sediment and a cloudy colorless low-viscosity oil.

### Production Example 2

### Synthesis of Polyether-Modified Silicone No. 2

187.6 g of a methylhydrogenpolysiloxane with the average compositional formula MD₄₀₀D^{H}₁₀M, 53.0 g allyl polyether with the average structural formula CH₂═CH—CH₂—O(C₂H₄O)₁₉(C₃H₆O)₁₉H, and 120 g IPA were introduced into a reactor and were heated to 55° C. while stirring under a nitrogen current. 0.055 g of an IPA solution of a platinum-1,3-divinyl-1,1,3,3-tetramethyldisiloxane complex, wherein the Pt concentration in the IPA solution was 4.5 weight %, was added and a reaction was run for 2.5 hours at 80° C. 2 g of the reaction solution was then recovered and checked by gas production by alkali decomposition, which showed that the conversion had reached the target range of 40%±5%. This was followed by the addition of 0.055 g of the previously indicated platinum catalyst and 30.7 g of a vinyl-monoterminated dimethylpolysiloxane with the structural formula CH₂═CHSiMe₂(OSiMe₂)₆OSiMe₃ and reaction for 3 hours at 80° C. 2 g of the reaction solution was recovered and checked by gas production by alkali decomposition, which showed that the reaction was completed.

This reaction solution was diluted by the addition thereto of 0.08 g vitamin E and 285 g dimethylpolysiloxane (2 mm²/s, 25° C.) with mixing. The low-boiling fraction other than the diluent was distilled out by heating the dilution under reduced pressure to obtain a mixture 2 comprising the chain-form dimethylpolysiloxane (2 mm²/s, 25° C.) and a composition containing a polyether group- and branched linear siloxane structure-containing organopolysiloxane with the average structural formula MD₄₀₀D^{R}*⁴¹₆D^{R}*²¹₄M; this polyether group- and branched linear siloxane structure-containing organopolysiloxane is referred to below as polyether-modified silicone No. 2 and had an HLB value of 1.6. The polyether-modified silicone:diluent ratio in mixture 2 was 40:60.

R*⁴¹ in the preceding formula indicates —C₂H₄SiMe₂(OSiMe₂)₆OSiMe₃.

R*²¹ in the preceding formula is defined as above.

Mixture 2 was a uniform, grayish-brown viscous liquid; after storage for two months at room temperature, a distinct sediment could not be seen, but with regard to the appearance the perception of nonuniformity had increased.

### Production Example 3

### Synthesis of Polyether-Modified Silicone No. 3

224.6 g of a methylhydrogenpolysiloxane with the average compositional formula MD₄₀₀D^{H}₁₀M, 63.7 g allyl polyether with the average structural formula CH₂═CH—CH₂—O(C₂H₄O)₁₉(C₃H₆O)₁₉H, 15.4 g of the vinyltristrimethylsiloxysilane with the average structural formula CH₂═CH—Si(OSiMe₃)₃, 94 g IPA, and 0.60 g of a 1.5 weight % methanolic sodium acetate solution were introduced into a reactor and were heated to 55° C. while stirring under a nitrogen current. 0.80 g of a 1 weight % IPA solution of chloroplatinic acid was added and a reaction was run for 5 hours at 80° C. 2 g of the reaction solution was then recovered and the completion of the reaction was confirmed through gas production by alkali decomposition.

This reaction solution was diluted by the addition thereto of 0.12 g vitamin E and 427 g dimethylpolysiloxane (2 mm²/s, 25° C.) with mixing. The low-boiling fraction other than the diluent was distilled out by heating the dilution under reduced pressure to obtain a mixture 3 comprising the chain-form dimethylpolysiloxane (2 mm²/s, 25° C.) and a composition containing a polyether group- and siloxane dendron branched structure-containing organopolysiloxane with the average compositional formula MD₄₀₀D^{R}*³¹₆D^{R}*²¹₄M; this polyether group- and siloxane dendron branched structure-containing organopolysiloxane is referred to below as polyether-modified silicone No. 3 and had an HLB value of 1.7. The polyether-modified silicone:diluent ratio in mixture 3 was 40:60.

R*³¹ in the preceding formula indicates —C₂H₄Si(OSiMe₃)₃.

R*²¹ in the preceding formula indicates —C₃H₆O(C₂H₄O)₁₉(C₃H₆O)₁₉H.

Mixture 3 was a uniform, whitish-brown viscous liquid from immediately after production to after storage for 2 months at room temperature. However, after 4 months, while a distinct sediment was not present, with regard to the appearance the perception of nonuniformity had increased.

The average compositional formulas and other properties are reported below for the “polyether-modified silicone No. 1”, “polyether-modified silicone No. 2”, and “polyether-modified silicone No. 3” according to the present invention and synthesized by the previously described methods.

TABLE 1

| silicone | average compositional | | |
|---|---|---|---|
| compound | formula | HLB | state |
| composition | MD₄₀₆D^{R*21}₄M | | whitish-brown viscous |
| containing | *diluted to a 40 mass % | 1.8 | liquid (phase separation |
| polyether- | concentration with | | and sedimentation after |
| modified | dimethylpolysiloxane | | two months) |
| silicone No. 1 | | | |
| composition | MD₄₀₀D^{R*41}₆D^{R*21}₄M | 1.6 | grayish-brown viscous |
| containing | *diluted to a 40 mass % | | liquid (after two months, |
| polyether- | concentration with | | a distinct sediment was not |
| modified | dimethylpolysiloxane | | present, but the perception |
| silicone No. 2 | | | of nonuniformity had |
| | | | increased) |
| composition | MD₄₀₀D^{R*31}₆D^{R*21}₄M | 1.7 | whitish-brown viscous |
| containing | *diluted to a 40 mass % | | liquid (after four months, |
| polyether- | concentration with | | a distinct sediment was |
| modified | dimethylpolysiloxane | | not present, but the |
| silicone No. 3 | | | perception of |
| | | | nonuniformity had |
| | | | increased) |

The structure and classification of the functional groups referenced in the table are given below.

<Hydrophilic Group: R*>

R*²¹═C₃H₆O(C₂H₄O)₁₉(C₃H₆O)₁₉H

<Siloxane Dendron Branched Structure-Containing Group: R*³>

R*³¹═C₂H₄Si(OSiMe₃)₃

<Branched Linear Polysiloxane Structure-Containing Group: R*⁴>

R*⁴¹═C₂H₄SiMe₂(OSiMe₂)₆OSiMe₃

The various oils investigated as compatibilizing agents for the examples are given in the following table. These were all liquids at 30° C.

TABLE 2

| | | average | | | |
|---|---|---|---|---|---|
| | | number of | | number | |
| | | hydroxyl | | of moles | |
| | | groups | | of oxy- | average |
| | | in each | | ethylene | molecular |
| abbreviation | name | molecule | HLB | addition | weight |
| ISA | isostearyl alcohol | 1 | 1.3 | 0 | 270 |
| ISG | glyceryl monoisostearate | 2 | 4.0 | 0 | 358 |
| ISS | sorbitan monoisostearate | 3 | 5.0 | 0 | 430 |
| ISP | propylene glycol | 1 | 1.0 | 0 | 342 |
| | isostearate | | | | |
| PPGS | stearyl PPG-15 | 1 | 0.3 | 0 | 1140 |
| ISGE | isostearyl glyceryl ether | 2 | 2.0 | 0 | 344 |
| PPGM | PPG-3 myristyl ether | 1 | 0.9 | 0 | 388 |
| SMO | sorbitan monooleate | 3 | 4.3 | 0 | 428 |
| SSO | sorbitan sesquioleate | 2.5 | 3.7 | 0 | 561 |
| STO | sorbitan trioleate | 1 | 1.8 | 0 | 960 |
| OMG | oleoyl monoglyceride | 2 | 2.8 | 0 | 356 |
| LC 4-2737 | silanol-modified silicone *1) | 2 | 1.0 | 0 | 700 |
| SF 8428 | alcohol-modified silicone *2) | 3 | 0.2 | 0 | 6000 |
| F2-276-01 | alcohol-modified silicone *3) | 5.4 | 1.8 | 1 | 2700 |
| BY 16-799 | phenol-modified silicone *4) | 2 | 0.5 | 0 | 1400 |

Note
*1): LC 4-2737 is a dimethylpolysiloxane modified by SiOH at both terminals, having a viscosity of 40 mm²/s and produced by the Dow Corning Corporation.
*2): SF 8428 is a PPG-2 dimethicone having a viscosity of 160 mm²/s and produced by Dow Corning Toray Co., Ltd.
*3): F2-276-01 is a dimethylsiloxane · methyl(3-(2-hydroxyethyl)propyl ether)siloxane copolymer having a viscosity of 200 mm²/s and produced by Dow Corning Toray Co., Ltd.
*4): BY16-799 is a dimethylpolysiloxane modified by allylphenol at both terminals, having a viscosity of 80 mm²/s and produced by Dow Corning Toray Co., Ltd.

The various oils investigated as compatibilizing agents for the comparative examples are given in the Table 3 below. These were all liquids at 25° C.

TABLE 3

| | | average | | | |
|---|---|---|---|---|---|
| | | number | | | |
| | | of | | number | |
| | | hydroxyl | | of moles | |
| | | groups | | of oxy- | average |
| abbrevi- | | in each | | ethylene | molecular |
| ation | name | molecule | HLB | addition | weight |
| IPM | isopropyl myristate | 0 | 0 | 0 | 270 |
| IPP | isopropyl palmitate | 0 | 0 | 0 | 298 |
| IOTG | glyceryl tri-2- | 0 | 0 | 0 | 470 |
| | ethylhexanoate | | | | |
| CCTG | glyceryl | 0 | 0 | 0 | 290 |
| | tri(caprylate/caprate) | | | | |
| ININ | isononyl isononanoate | 0 | 0 | 0 | 284 |
| HDEH | hexyldecyl | 0 | 0 | 0 | 368 |
| | ethylhexanoate | | | | |
| IDNP | isodecyl neopentanoate | 0 | 0 | 0 | 242 |
| EPDNP | diethylpentanediol | 0 | 0 | 0 | 328 |
| | dineopentanoate | | | | |
| K-230 | mineral oil | 0 | 0 | 0 | *7) |
| ID | isododecane | 0 | 0 | 0 | 170 |
| IP | hydrogenated | 0 | 0 | 0 | *8) |
| | polyisobutene | | | | |
| EtOH | ethanol | 1 | 7.4 | 0 | 46 |
| PG | propylene glycol | 2 | 8.9 | 0 | 76 |
| BG | 1,3-butylene glycol | 2 | 7.6 | 0 | 90 |
| DPG | dipropylene glycol | 2 | 5.1 | 0 | 134 |
| H2O | water | 1.5 | 20 | 0 | 18 |
| SH 556 | phenyltrimethicone | 0 | 0 | 0 | 372 |
| 2cst | dimethylpolysiloxane | 0 | 0 | 0 | 384 |
| | (2 mm²/s) | | | | |
| 6cst | dimethylpolysiloxane | 0 | 0 | 0 | 754 |
| | (6 mm²/s) | | | | |
| FZ-77 | polyether-modified | 0 | 10 | 7 | 602 |
| | silicone *9) | | | | |
| SS-2910 | PEG-10 dimethicone | 2 | 3.8 | 10 | 4600 |

Note
*7): Average molecular weight unknown. Viscosity = 13.7 mm²/s at 37.8° C.
*8): Average molecular weight unknown. Viscosity = 3.1 mm²/s at 37.8° C.
*9): 1,1,1,3,5,5,5-heptamethyl-3-(propyl(poly(EO))methyl)trisiloxane

### Examples 1 to 45 and Comparative Examples 1 to 66

### Investigation of the Compatibilization of Mixtures 1 to 3 by Various Oils

Using the procedure given below, each oil was added at two levels, 20% and 5%, to each of the mixtures 1 to 3 obtained in the previously described Production Examples 1 to 3; the polyether-modified silicone/2cst dimethylpolysiloxane ratio in these mixtures 1 to 3 was 40/60. The resulting samples were evaluated using the criteria indicated below.

Production and Test Procedures

1. The particular mixture 1 to 3 and the particular oil were introduced into a 150-mL plastic wide-mouth ointment jar to provide a total quantity of 20.0 g; this was placed in a dental mixer (MIGMA Mikrona mixer from Mikrona Technologie AG) and stirred by shaking for 36 seconds.
2. The plastic jar was removed from the mixer and the contents were distributed in three equal portions into 20-mL vials.
3. The appearance of the liquid was recorded.
4. The vials were held at quiescence for 2 months in a thermostat at 0° C., 25° C., or 40° C., after which the appearance of the contents was recorded for each temperature. Because some samples stored at 0° C. also underwent solidification, the appearance was scored after returning to 25° C.; this is indicated as “0 [symbol] 25° C.” in the tables.

Formulations

mixture 1 to 3/oil=16.0 g/4.0 g(20% compatibilizer incorporation) formulation A

mixture 1 to 3/oil=19.0 g/1.0 g(5% compatibilizer incorporation) formulation B

Evaluation

### 1. Appearance·State

++: uniform and almost transparent liquid, excellent fluidity

+: uniform and semi-transparent liquid, excellent fluidity

Δ: uniform and strongly turbid liquid, fluidity somewhat reduced

x: nonuniform liquid, or phase separation has occurred, or fluidity is absent (gelation)

Results of the Evaluations

Tables 4 and 5 report the evaluation results for the investigation of the compatibilization of mixture 1 using the various oils.
Tables 6 and 7 report the evaluation results for the investigation of the compatibilization of mixture 2 using the various oils.
Tables 8 and 9 report the evaluation results for the investigation of the compatibilization of mixture 3 using the various oils.

TABLE 4
Examples 1 to 15: Investigation of the improvement in
compatibility for the mixture of Production Example 1

| | | | Appearance | | | | overall |
|---|---|---|---|---|---|---|---|
| | compat- | formu- | | | | (0 [symbol] | evalu- |
| No. | ibilizer | lation | initial | (40° C.) | (25° C.) | 25° C.) | ation |
| 1 | ISA | A | + + | + + | + + | + + | + - + + |
| | | B | + | Δ | + | + + | |
| 2 | ISG | A | Δ - + | Δ - + | Δ - + | Δ - + | + |
| | | B | + + | + + | + + | + + | |
| 3 | ISS | A | + | Δ | + | + | + - + + |
| | | B | + + | + - + + | + + | + + | |
| 4 | ISP | A | + + | + + | + + | + + | + + |
| | | B | + + | + | + + | + + | |
| 5 | PPGS | A | x | + + | x | x | Δ - + |
| | | B | + + | Δ | + + | Δ - + | |
| 6 | ISGE | A | x | x | x | x | Δ - + |
| | | B | + + | + + | + + | + + | |
| 7 | PPGM | A | + + | + + | + + | + + | + + |
| | | B | + + | + + | + + | + + | |
| 8 | SMO | A | Δ - + | x - Δ | Δ - + | Δ - + | + |
| | | B | + + | + - + + | + + | + + | |
| 9 | SSO | A | + | Δ | + | Δ - + | + - + + |
| | | B | + + | + - + + | + + | + + | |
| 10 | STO | A | x | x | x | x | Δ - + |
| | | B | + + | + + | + + | + + | |
| 11 | OMG | A | + + | + + | + + | + | + + |
| | | B | + + | + + | + + | + + | |
| 12 | LC | A | x | x | x | x | Δ - + |
| | 4-2737 | B | + + | + + | + + | + + | |
| 13 | SF | A | Δ - + | Δ - + | Δ - + | Δ - + | Δ - + |
| | 8428 | B | + | Δ - + | + | + | |
| 14 | F2- | A | x | Δ | x | x - Δ | Δ |
| | 276-01 | B | + | Δ | + | Δ | |
| 15 | BY | A | + + | + + | + + | + + | + - + + |
| | 16-799 | B | + + | Δ - + | + + | + | |

TABLE 5
Comparative Examples 1 to 22: Investigation of the improvement in
compatibility for the mixture of Production Example 1

| | | | Appearance | | | | overall |
|---|---|---|---|---|---|---|---|
| | compat- | formu- | | | | (0 [symbol] | evalu- |
| No. | ibilizer | lation | initial | (40° C.) | (25° C.) | 25° C.) | ation |
| 1 | none | — | Δ | x | x | Δ | x - Δ |
| 2 | IPM | A | x | x | x | x | x |
| | | B | x | x | x | x | |
| 3 | IPP | A | x | x | x | x | x |
| | | B | x | x | x | x | |
| 4 | IOTG | A | x | x | x | x | x - Δ |
| | | B | Δ | x - Δ | Δ | Δ | |
| 5 | CCTG | A | x | x | x | x | x |
| | | B | x | x | x | x | |
| 6 | ININ | A | x | x | x | x | x |
| | | B | x | x | x | x | |
| 7 | HDEH | A | x | x | x | x | x |
| | | B | x | x | x | x | |
| 8 | IDNP | A | x | x | x | x | x |
| | | B | x | x | x | x | |
| 9 | EPDNP | A | Δ | x | x | x | x |
| | | B | x | x | x | x | |
| 10 | K-230 | A | x - Δ | x - Δ | x - Δ | x - Δ | x - Δ |
| | | B | x - Δ | x - Δ | x - Δ | x - Δ | |
| 11 | ID | A | x | x | x | x | x |
| | | B | x | x | x | x | |
| 12 | IP | A | x - Δ | x - Δ | x - Δ | x - Δ | x - Δ |
| | | B | x - Δ | x - Δ | x - Δ | x - Δ | |
| 13 | EtOH | A | x | x | x | x | x |
| | | B | Δ | x | x | Δ | |
| 14 | PG | A | x (gel) | x (gel) | x (gel) | x (gel) | x |
| | | B | x (gel) | x (gel) | x (gel) | x (gel) | |
| 15 | BG | A | x (gel) | x (gel) | x (gel) | x (gel) | x |
| | | B | x (gel) | x (gel) | x (gel) | x (gel) | |
| 16 | DPG | A | x (gel) | x (gel) | x (gel) | x (gel) | x |
| | | B | x (gel) | x (gel) | x (gel) | x (gel) | |
| 17 | H2O | A | x (gel) | x (gel) | x (gel) | x (gel) | x |
| | | B | x (gel) | x (gel) | x (gel) | x (gel) | |
| 18 | SH 556 | A | x | x | x | x | x |
| | | B | x | x | x | x | |
| 19 | 2cst | A | Δ | x | x | Δ | x - Δ |
| | | B | Δ | x | x | Δ | |
| 20 | 6cst | A | Δ | x | x | Δ | x - Δ |
| | | B | Δ | x | x | Δ | |
| 21 | FZ-77 | A | x | x | x | x | x |
| | | B | x | x | x | x | |
| 22 | SS- | A | Δ - + | x | x - Δ | Δ | x - Δ |
| | 2910 | B | x | x | x | x | |

TABLE 6
Examples 16 to 30: Investigation of the improvement in
compatibility for the mixture of Production Example 2

| | | | Appearance | | | | overall |
|---|---|---|---|---|---|---|---|
| | compat- | formu- | | | | (0 [symbol] | evalu- |
| No. | ibilizer | lation | initial | (40° C.) | (25° C.) | 25° C.) | ation |
| 16 | ISA | A | + + | + | + + | + + | + - + + |
| | | B | + | Δ | + | + + | |
| 17 | ISG | A | Δ - + | Δ - + | Δ - + | Δ - + | + |
| | | B | + + | + + | + + | + + | |
| 18 | ISS | A | + | Δ | + | + | + - + + |
| | | B | + + | + - + + | + + | + + | |
| 19 | ISP | A | + + | + + | + + | + + | + + |
| | | B | + + | + | + + | + + | |
| 20 | PPGS | A | x | + + | x | x | Δ - + |
| | | B | + + | Δ | + + | Δ - + | |
| 21 | ISGE | A | x - Δ | x | x - Δ | x - Δ | Δ - + |
| | | B | + + | + + | + + | + + | |
| 22 | PPGM | A | + + | + + | + + | + + | + + |
| | | B | + + | + + | + + | + + | |
| 23 | SMO | A | Δ - + | x - Δ | Δ - + | Δ - + | + |
| | | B | + + | + - + + | + + | + + | |
| 24 | SSO | A | + | Δ | + | + | + - + + |
| | | B | + + | + - + + | + + | + + | |
| 25 | STO | A | x | x | x | x | Δ - + |
| | | B | + + | + + | + + | + + | |
| 26 | OMG | A | + + | + + | + + | + | + + |
| | | B | + + | + + | + + | + + | |
| 27 | LC | A | x | x | x | x | Δ |
| | 4-2737 | B | + | + | + | + | |
| 28 | SF | A | + | + | + | + | + |
| | 8428 | B | + | + | + | + | |
| 29 | F2- | A | x | Δ | x | x - Δ | Δ |
| | 276-01 | B | + | Δ | + | Δ | |
| 30 | BY | A | + + | + + | + + | + + | + - + + |
| | 16-799 | B | + + | + | + + | + | |

TABLE 7
Comparative Examples 23 to 44: Investigation of the improvement in
compatibility for the mixture of Production Example 2

| | | | Appearance | | | | overall |
|---|---|---|---|---|---|---|---|
| | compat- | formu- | | | | (0 [symbol] | evalu- |
| No. | ibilizer | lation | initial | (40° C.) | (25° C.) | 25° C.) | ation |
| 23 | none | — | Δ | x | x - Δ | Δ | x - Δ |
| 24 | IPM | A | x | x | x | x | x |
| | | B | x | x | x | x | |
| 25 | IPP | A | x | x | x | x | x |
| | | B | x | x | x | x | |
| 26 | IOTG | A | Δ | x | Δ | Δ | x - Δ |
| | | B | Δ | x - Δ | Δ | Δ | |
| 27 | CCTG | A | Δ | x | x - Δ | Δ | x |
| | | B | x | x | x | x | |
| 28 | ININ | A | Δ | x | x - Δ | Δ | x |
| | | B | x | x | x | x | |
| 29 | HDEH | A | Δ | x | x - Δ | Δ | x |
| | | B | x | x | x | x | |
| 30 | IDNP | A | Δ | x | x - Δ | Δ | x |
| | | B | x | x | x | x | |
| 31 | EPDNP | A | Δ | x | x - Δ | Δ | x |
| | | B | x | x | x | x | |
| 32 | K-230 | A | x - Δ | x - Δ | x - Δ | x - Δ | x - Δ |
| | | B | x - Δ | x - Δ | x - Δ | x - Δ | |
| 33 | ID | A | x - Δ | x | x | x - Δ | x |
| | | B | x - Δ | x | x | x - Δ | |
| 34 | IP | A | x - Δ | x - Δ | x - Δ | x - Δ | x - Δ |
| | | B | x - Δ | x - Δ | x - Δ | x - Δ | |
| 35 | EtOH | A | x | x | x | x | x |
| | | B | Δ | x | x | Δ | |
| 36 | PG | A | x (gel) | x (gel) | x (gel) | x (gel) | x |
| | | B | x (gel) | x (gel) | x (gel) | x (gel) | |
| 37 | BG | A | x (gel) | x (gel) | x (gel) | x (gel) | x |
| | | B | x (gel) | x (gel) | x (gel) | x (gel) | |
| 38 | DPG | A | x (gel) | x (gel) | x (gel) | x (gel) | x |
| | | B | x (gel) | x (gel) | x (gel) | x (gel) | |
| 39 | H2O | A | x (gel) | x (gel) | x (gel) | x (gel) | x |
| | | B | x (gel) | x (gel) | x (gel) | x (gel) | |
| 40 | SH 556 | A | x | x | x | x | x |
| | | B | x | x | x | x | |
| 41 | 2cst | A | Δ | x | x | Δ | x - Δ |
| | | B | Δ | x | x - Δ | Δ | |
| 42 | 6cst | A | Δ | x | x - Δ | Δ | x - Δ |
| | | B | Δ | x | x - Δ | Δ | |
| 43 | FZ-77 | A | x - Δ | x | x | x - Δ | x |
| | | B | x | x | x | x | |
| 44 | SS- | A | Δ - + | x | x - Δ | Δ | x - Δ |
| | 2910 | B | x | x | x | x | |

TABLE 8
Examples 31 to 45: Investigation of the improvement in
compatibility for the mixture of Production Example 3

| | | | Appearance | | | | overall |
|---|---|---|---|---|---|---|---|
| | compat- | formu- | | | | (0 [symbol] | evalu- |
| No. | ibilizer | lation | initial | (40° C.) | (25° C.) | 25° C.) | ation |
| 31 | ISA | A | + + | + + | + + | + + | + + |
| | | B | + - + + | + | + - + + | + + | |
| 32 | ISG | A | + | + | + | + | + - + + |
| | | B | + + | + + | + + | + + | |
| 33 | ISS | A | + - + + | Δ - + | + - + + | + - + + | + - + + |
| | | B | + + | + + | + + | + + | |
| 34 | ISP | A | + + | + + | + + | + + | + + |
| | | B | + + | + - + + | + + | + + | |
| 35 | PPGS | A | x - Δ | + + | x - Δ | x - Δ | Δ - + |
| | | B | + + | Δ - + | + + | + | |
| 36 | ISGE | A | x - Δ | x - Δ | x - Δ | x - Δ | Δ - + |
| | | B | + + | + + | + + | + + | |
| 37 | PPGM | A | + + | + + | + + | + + | + + |
| | | B | + + | + + | + + | + + | |
| 38 | SMO | A | + | Δ | + | + | + - + + |
| | | B | + + | + + | + + | + + | |
| 39 | SSO | A | + - + + | Δ - + | + - + + | + | + - + + |
| | | B | + + | + + | + + | + + | |
| 40 | STO | A | x - Δ | x - Δ | x - Δ | x - Δ | Δ - + |
| | | B | + + | + + | + + | + + | |
| 41 | OMG | A | + + | + + | + + | + | + + |
| | | B | + + | + + | + + | + + | |
| 42 | LC | A | x - Δ | x - Δ | x - Δ | x - Δ | Δ - + |
| | 4-2737 | B | + + | + + | + + | + + | |
| 43 | SF | A | + | + | + | + | + |
| | 8428 | B | + - + + | + | + - + + | + - + + | |
| 44 | F2- | A | x - Δ | Δ - + | x - Δ | Δ | Δ - + |
| | 276-01 | B | + - + + | Δ - + | + - + + | Δ - + | |
| 45 | BY | A | + + | + + | + + | + + | + + |
| | 16-799 | B | + + | + | + + | + - + + | |

TABLE 9
Comparative Examples 45 to 66: Investigation of the improvement in
compatibility for the mixture of Production Example 3

| | | | Appearance | | | | overall |
|---|---|---|---|---|---|---|---|
| | compat- | formu- | | | | (0 [symbol] | evalu- |
| No. | ibilizer | lation | initial | (40° C.) | (25° C.) | 25° C.) | ation |
| 45 | none | — | Δ | Δ | Δ | Δ | Δ |
| 46 | IPM | A | x | x | x | x | x |
| | | B | x | x | x | x | |
| 47 | IPP | A | x | x | x | x | x |
| | | B | x | x | x | x | |
| 48 | IOTG | A | x | x | x | x | x - Δ |
| | | B | Δ | Δ | Δ | Δ | |
| 49 | CCTG | A | x | x | x | x | x |
| | | B | x | x | x | x | |
| 50 | ININ | A | x | x | x | x | x |
| | | B | x | x | x | x | |
| 51 | HDEH | A | x | x | x | x | x |
| | | B | x | x | x | x | |
| 52 | IDNP | A | x | x | x | x | x |
| | | B | x | x | x | x | |
| 53 | EPDNP | A | Δ | x - Δ | x - Δ | x | x |
| | | B | x | x | x | x | |
| 54 | K-230 | A | x - Δ | x - Δ | x - Δ | x - Δ | x - Δ |
| | | B | x - Δ | x - Δ | x - Δ | x - Δ | |
| 55 | ID | A | x | x | x | x | x |
| | | B | x | x | x | x | |
| 56 | IP | A | x - Δ | x - Δ | x - Δ | x - Δ | x - Δ |
| | | B | x - Δ | x - Δ | x - Δ | x - Δ | |
| 57 | EtOH | A | x | x | x | x | x |
| | | B | Δ | x | x | Δ | |
| 58 | PG | A | x (gel) | x (gel) | x (gel) | x (gel) | x |
| | | B | x (gel) | x (gel) | x (gel) | x (gel) | |
| 59 | BG | A | x (gel) | x (gel) | x (gel) | x (gel) | x |
| | | B | x (gel) | x (gel) | x (gel) | x (gel) | |
| 60 | DPG | A | x (gel) | x (gel) | x (gel) | x (gel) | x |
| | | B | x (gel) | x (gel) | x (gel) | x (gel) | |
| 61 | H2O | A | x (gel) | x (gel) | x (gel) | x (gel) | x |
| | | B | x (gel) | x (gel) | x (gel) | x (gel) | |
| 62 | SH | A | x | x | x | x | x |
| | 556 | B | x | x | x | x | |
| 63 | 2cst | A | Δ | Δ | Δ | Δ | Δ |
| | | B | Δ | Δ | Δ | Δ | |
| 64 | 6cst | A | Δ | Δ | Δ | Δ | Δ |
| | | B | Δ | Δ | Δ | Δ | |
| 65 | FZ-77 | A | x | x | x | x | x |
| | | B | x | x | x | x | |
| 66 | SS- | A | Δ - + | x - Δ | Δ | Δ - + | x - Δ |
| | 2910 | B | x | x | x | x | |

A comparison of the compatibility test results shown in Tables 4, 6, and 8 for examples according to the present application with the compatibility test results shown in Tables 5, 7, and 9 for comparative examples clearly demonstrates a superior compatibility for the cosmetic raw materials that used the compatibilizers provided in Table 2 as examples in the present application. In addition, the compatibility test results in the examples, which are given in Tables 4, 6, and 8, are excellent for all of the mixtures from Production Examples 1 to 3, even though the polyether-modified silicones had different structures.

Based on these results, the oils (C) designated in Table 2, i.e., oils that were a liquid at 30° C. and that had at least one hydroxyl group in each molecule, an HLB in the range from 0.1 to 6.0, an average molecular weight in the range from 200 to 7000, and from 0 to 3 moles of oxyethylene addition, were more effective as compatibilizers of mixtures of (A) a polyether-modified silicone and (B) a chain-form silicone oil than were the other oils described in Table 3 and were able at a small quantity of addition to provide improvement to give a stable liquid that had a semi-transparent to transparent appearance. Accordingly, a mixture comprising components (A), (B), and (C) is a practical cosmetic raw material because it resists phase separation with elapsed time.

### Example 46 and Comparative Example 67

### Incorporation into a Cosmetic

The specific usefulness of the cosmetic raw material according to the present application is shown in the following through incorporation as a raw material in an actual cosmetic. For the evaluation of incorporation, a water-in-oil cosmetic (“W/O cream”) was selected, as this is a typical formulation in which the cyclic silicone D5 is incorporated. Using the compositions in Table 10, the results are shown below for the preparation and evaluation of W/O creams in an example and a comparative example. The FV-1027-99 used in Comparative Example 67 is a cosmetic raw material that employs D5 as a diluent.

TABLE 10
Example 46 and Comparative Example 67: W/O creams

| | | Example | Comparative |
|---|---|---|---|
| | component, weight % | 46 | Example 67 |
| oil | methylphenylpolysiloxane *1) | 5.0 | 5.0 |
| phase | dimethylpolysiloxane (2 mm²/s) | 18.5 | — |
| | decamethylcyclopentasiloxane (D5) | — | 18.5 |
| | formulation A mixture from | 2.5 | — |
| | Example 1 | | |
| | FV-1027-99 *2) | — | 2.5 |
| aqueous | glycerol | 5.0 | 5.0 |
| phase | methyl para-hydroxbenzoate | 0.1 | 0.1 |
| | sodium chloride | 1.0 | 1.0 |
| | purified water | balance | balance |

Note
*1): SH546, a phenyltrimethicone from Dow Corning Toray Co., Ltd.
*2): A mixture obtained by production according to “Synthesis of polyether-modified silicone No. 1” of Production Example 1 using decamethylcyclopentasiloxane in place of the dimethylpolysiloxane (2 mm²/s, 25° C.) that was used as the diluent. The polyether-modifiedsilicone:diluent ratio in this mixture was 40:60.

Production Procedure

1. The oil phase and aqueous phase were each weighed into containers and were dissolved to uniformity at 70° C.

2. The oil phase was placed in a Homo Disper and the aqueous phase was poured in at an approximately constant rate over approximately 40 seconds while stirring at 1000 rpm.

3. Emulsification and dispersion were performed by stirring for 5 minutes at 3000 rpm followed by cooling to about 30° C. to give the W/O cream.

Evaluation Results

The emulsions of Example 46 and Comparative Example 67 exhibited the same sensory characteristics, i.e., they both had a finely textured appearance and exhibited little oiliness and no stickiness when applied on the forearm. In addition, after storage stability testing for 1 month at 40° C., both emulsions were free of problems with regard to the state of the emulsion and uniformity of appearance and thus exhibited the same stability.

The results of this evaluation confirmed that the use of the cosmetic raw material according to the present application, i.e., the use of a raw material that employs a chain-form silicone oil, can provide a cosmetic that has the same sensory characteristics and stability as for the use of a cyclic silicone.

### Formulation Examples 1 to 9

### Formulation Examples that Incorporate the Cosmetic Raw Material of the Present Invention

Specific formulation examples of cosmetics and topicals that incorporate the cosmetic raw material according to the present invention are described below in order to further illustrate the utility of the cosmetic raw material according to the present invention; however, cosmetics that can incorporate the cosmetic raw material according to the present invention are of course not limited to the types and compositions described in these formulation examples. The cosmetic raw materials indicated with product numbers in the formulation examples in all instances refer to the names of products sold by Dow Corning Toray Co., Ltd. The viscosity in the formulation examples is the kinematic viscosity measured at 25° C. in units of mm²/s. The cosmetic raw material according to the present invention is indicated using the phrase, “formulation A mixture from Example ˜”. However, in Formulation Example 9 alone, a formulation example is provided that uses the W/O cream of Example 46 as a sunscreen base.

### Formulation Example 1

### Emulsified Foundation

### Components

| | | |
|---|---|---|
| 1. | dimethylpolysiloxane (2 mm²/s) | 12.0 parts |
| 2. | sorbitan monoisostearate | 0.7 part |
| 3. | diglyceryl monoisostearate | 0.7 part |
| 4. | formulation A mixture from Example 1 | 2.5 parts |
| 5. | liquid paraffin | 5.0 parts |
| 6. | vaseline | 1.5 parts |
| 7. | methylphenylpolysiloxane | 4.0 parts |
| | (product number: SH546) | |
| 8. | trimethylsiloxysilicic acid | 1.0 part |
| 9. | 2-ethylhexyl para-methoxycinnamate | 3.0 parts |
| 10. | purified water | 48.3 parts |
| 11. | distearyldimethylammonium chloride | 0.05 part |
| 12. | sorbitol | 3.0 parts |
| 13. | magnesium sulfate | 0.1 part |
| 14. | paraben | 0.15 part |
| 15. | ethanol | 5.0 parts |
| 16. | rutile titanium oxide | 10.0 parts |
| 17. | iron oxide | 0.5 part |
| 18. | talc | 2.5 parts |

### Production Method

Heat components 1 to 9 (oil phase components) to 70° C. and disperse therein components 16 to 18 (powder components). While stirring the result, add components to 14 (aqueous phase components), which have been pre-heated to 70° C.; emulsify; then cool to room temperature. Finally, add component 15 and stir to obtain the intended emulsified foundation.

### Effects

This is a stable powder-containing emulsified cosmetic that resists separation and aggregation. In addition, the smoothly spreadable, stickiness-free use sensation is persistent.

### Formulation Example 2

### Sunscreen

### Components

| | | |
|---|---|---|
| 1. | sorbitan monoisostearate | 1.0 part |
| 2. | formulation A mixture from Example 31 | 1.6 parts |
| 3. | methyltrimethicone | 15.0 parts |
| 4. | caprylylmethicone (product number: | 9.0 parts |
| | SS-3408) | |
| 5. | methylphenylpolysiloxane (product | 2.0 parts |
| | number: SH556) | |
| 6. | 2-ethylhexyl para-methoxycinnamate | 6.9 parts |
| 7. | trimethylsiloxysilicic acid | 2.0 parts |
| 8. | liquid silk extract | 0.5 part |
| 9. | Peony root extract | 0.5 part |
| 10. | sage extract | 0.5 part |
| 11. | 1,3-butylene glycol | 2.0 parts |
| 12. | ethanol | 8.0 parts |
| 13. | para-hydroxybenzoate ester | suitable quantity |
| 14. | purified water | 47.0 parts |
| 15. | microparticulate titanium oxide | 3.0 parts |
| 16. | silicone resin-coated silicone | 1.0 part |
| | rubber powder | |

### Production Method

Mix components 1 to 7 (oil phase components) and dissolve by heating to 80° C. and add components 15 and 16 (powder components) thereto and disperse. While stirring the result, add components 8 to 11, 13, and 14 (aqueous phase components), which have been pre-heated to 80° C. and dissolved; emulsify; then cool to room temperature. Finally, add component 12 and stir to obtain the intended sunscreen.

### Effects

An excellent post-application finish is obtained that hides wrinkles and skin texture; an excellent use sensation, with a light spreadability and free of a squeaky feel, is obtained. Separation of the aqueous phase or oil phase from the emulsion is inhibited and the storage stability is also excellent.

### Formulation Example 3

### Aerosol Hair Cream

### Components

| | | |
|---|---|---|
| 1. | dimethylpolysiloxane (2 mm²/s) | 2.0 parts |
| 2. | sorbitan monoisostearate | 0.3 part |
| 3. | diglyceryl monoisostearate | 0.2 part |
| 4. | formulation A mixture from | 1.5 parts |
| | Example 1 | |
| 5. | light liquid isoparaffin | 8.0 parts |
| 6. | liquid paraffin | 2.5 parts |
| 7. | 2-ethylhexyl para-methoxycinnamate | 2.0 parts |
| 8. | purified water | 26.5 parts |
| 9. | 1,3-butylene glycol | 1.0 part |
| 10. | polyvinylpyrrolidone | 0.5 part |
| 11. | ethanol | 4.0 parts |
| 12. | silicone powder (product number: | 1.5 parts |
| | Torayfil E-506S) | |
| 13. | LPG propellant | 40.0 parts |
| 14. | dimethyl ether propellant | 10.0 parts |

### Production Method

Heat components 1 to 7 (oil phase components) to 70° C. and disperse therein component 12 (powder component). While stirring the result, add components 8 to 10 (aqueous phase components), which have been pre-heated to 70° C.; emulsify; then cool to room temperature. Finally, add component 11 and stir to obtain an emulsified stock liquid. Fill this stock liquid into a pressure-resistant container; install the valve; and then fill with the propellant.

### Effects

The propellant redispersibility with elapsed time is excellent; the spray produced during spraying is uniform and fine; and the spreadability when used is also excellent.

### Formulation Example 4

### Make-Up Foundation

### Components

| | | |
|---|---|---|
| 1. | product number: DC9011 Silicone | 2.0 parts |
| | Elastomer Blend *1) | |
| 2. | product number: DC9041 Silicone | 1.0 part |
| | Elastomer Blend *2) | |
| 3. | formulation A mixture from Example 31 | 2.5 parts |
| 4. | dimethylpolysiloxane (2 mm²/s) | 20.0 parts |
| 5. | crosslinked silicone powder mixture | 3.0 parts |
| 6. | fluorosilicate fluid *3) | 10.0 parts |
| 7. | sorbitan monoisostearate | 1.0 part |
| 8. | 1,3-butylene glycol | 7.0 parts |
| 9. | cranberry extract | 0.3 part |
| 10. | sodium citrate | 0.5 part |
| 11. | purified water | 36.7 parts |
| 12. | ethanol | 5.0 parts |
| 13. | octylalkoxysilane-treated | 6.0 parts |
| | microparticulate titanium oxide | |
| 14. | iron oxide | 0.5 part |
| 15. | talc | 4.5 parts |

Note
*1): A decamethylcyclopentasiloxane dilution of a crosslinked polyether-modified silicone, containing 15% elastomer component.
Note
*2): A straight-chain dimethylsilicone (5 mm²/s) dilution of a crosslinked organopolysiloxane (dimethicone crosspolymer), containing 16% elastomer component.
Note
*3): A product produced according to the production method described in Production Example 1 of JP 09-012431; contains 50% hydroxyl-containing trifluoropropyldimethyltrimethylsiloxysilicic acid and 50% decamethylcyclopentasiloxane.

### Production Method

Add component 8 while mixing and stirring the pre-mixed-and-ground components 13 to 15 (powder components) with components 1 to 7 (oil phase components) using a Disper mixer. Gradually add a solution prepared from components 9 and 10 and a 16 mass % portion of component 11. While dispersing this mixture using a homomixer, add the remaining 20.7 mass % portion of component 11 and then add component 12 to produce the make-up foundation.

### Effects

A smooth use sensation is provided; an uncomfortable feel, such as dryness, is not produced with elapsed time; and the make-up durability is also excellent. Separation of the aqueous phase or oil phase from the emulsion is inhibited and the storage stability is also excellent.

### Formulation Example 5

### Cosmetic Foundation

### Components

| | | |
|---|---|---|
| 1. | product number: DC 2-1184 Fluid *4) | 16.9 parts |
| 2. | product number: 9041 Silicone Elastomer | 5.0 parts |
| | Blend *2) | |
| 3. | product number: DC 1503 Fluid *5) | 1.0 part |
| 4. | formulation A mixture from Example 1 | 3.75 parts |
| 5. | sorbitan sesquioleate | 1.5 parts |
| 6. | phenyltrimethicone (product number: SH556) | 10.0 parts |
| 7. | caprylylmethicone (product number: SS-3408) | 7.8 parts |
| 8. | methicone-coated iron oxide red | 0.59 part |
| 9. | methicone-coated iron oxide yellow | 1.22 parts |
| 10. | methicone-coated iron oxide black | 0.11 part |
| 11. | methicone-coated titanium dioxide | 3.56 parts |
| 12. | methicone-coated ultrafine titanium dioxide | 4.50 parts |
| 13. | purified water | 37.75 parts |
| 14. | 1,3-butylene glycol | 5.0 parts |
| 15. | xanthan gum | 0.10 part |
| 16. | magnesium sulfate | 1.00 part |
| 17. | polyoxyethylene (7 moles) lauryl ether | 0.25 part |

Note
*2): A straight-chain dimethylsilicone (5 mm²/s) dilution of a crosslinked organopolysiloxane (dimethicone crosspolymer), containing 16% elastomer component.
Note
*4): A blend of the trimer, tetramer, and pentamer of a straight-chain dimethylpolysiloxane endblocked by the trimethylsiloxy group at both molecular chain terminals.
Note
*5): A straight-chain dimethylsilicone (5 mm²/s) dilution of a high degree of polymerization dimethiconol gum, containing 12% dimethiconol component.

### Production Method

Components 1 to 7 (oil phase components), components 8 to 12 (pigment components), and components 13 to 17 are each preliminarily and separately prepped by low-shear mixing. These are then mixed with each other by high-shear blending. Specifically, the pigment phase is added to the oil phase and mixing is carried out at room temperature to provide a uniform mixture. The aqueous phase is then gradually added to the oil-pigment phase and homogenization to smoothness is performed.

### Effects

The coating behavior and tactile feel characteristics, e.g., cushioning behavior, body characteristics, and slip behavior, are excellent, and stickiness and an oily sensation are absent. In addition, separation of the aqueous phase or oil phase from the emulsion is inhibited and the storage stability is also excellent.

### Formulation Example 6

### Lipstick

### Components

| | | |
|---|---|---|
| 1. | candelilla wax | 7.0 parts |
| 2. | ceresin | 6.0 parts |
| 3. | vaseline | 10.0 parts |
| 4. | castor oil | 26.2 parts |
| 5. | isostearyl alcohol | 5.0 parts |
| 6. | isononyl isononanoate | 3.0 parts |
| 7. | caprylylmethicone (product | 2.0 parts |
| | number: SS-3408) | |
| 8. | lanolin | 10.0 parts |
| 9. | glyceryl trioctanoate | 15.0 parts |
| 10. | formulation A mixture from | 6.0 parts |
| | Example 31 | |
| 11. | Red No. 202 | 1.0 part |
| 12. | Red No. 201 | 2.0 parts |
| 13. | iron oxide black | 1.3 parts |
| 14. | titanium oxide | 1.5 parts |
| 15. | titanium mica | 4.0 parts |

### Production Method

Heat and dissolve components 1 to 10; add components 11 to 15 and mix to uniformity; then fill into a container to obtain the lipstick.

### Effects

There is no perception of stickiness during application, and the make-up durability and the perception of adherence are also excellent.

### Formulation Example 7

### Eye Shadow

### Components

| | | |
|---|---|---|
| 1. | microcrystalline wax | 5.0 parts |
| 2. | ceresin | 5.0 parts |
| 3. | vaseline | 20.0 parts |
| 4. | glyceryl trioctanoate | 23.8 parts |
| 5. | propylene glycol isostearate | 10.0 parts |
| 6. | formulation A mixture from | 6.0 parts |
| | Example 4 | |
| 7. | ultramarine | 0.8 part |
| 8. | iron oxide black | 1.2 parts |
| 9. | titanium oxide | 1.2 parts |
| 10. | mica | 5.0 parts |
| 11. | sericite | 22.0 parts |

### Production Method

Mix components 1 to 6 and heat to 90° C. and dissolve; then add components 7 to 11 and mix to uniformity. Fill into a mold and then cool to obtain an oil-based solid eye shadow.

### Effects

There is no perception of stickiness during application, and the make-up durability and the perception of adherence are also excellent.

### Formulation Example 8

### Sunscreen Cosmetic Cream

### Components

| | | |
|---|---|---|
| 1. | octyl hydroxystearate | 2.5 parts |
| 2. | decyl oleate | 2.5 parts |
| 3. | octyl para-dimethylaminobenzoate | 2.0 parts |
| 4. | glyceryl stearate | 1.0 part |
| 5. | stearic acid | 2.0 parts |
| 6. | myristyl myristate | 1.0 part |
| 7. | caprylylmethicone (product number: | 1.5 parts |
| | SS-3408) | |
| 8. | product number: FV-1034-05 *6) | 1.0 parts |
| 9. | formulation A mixture from Example 1 | 2.0 parts |
| 10. | purified water | 52.7 parts |
| 11. | glycerol | 5.0 parts |
| 12. | polyethylene glycol | 5.0 parts |
| 13. | tetrahydroxypropylethylenediamine | 0.5 part |
| 14. | amorphous finely divided titanium oxide | 5.0 parts |
| | composite *7) | |
| 15. | titanium oxide | 3.0 parts |
| 16. | sericite | 5.0 parts |
| 17. | bentonite | 3.0 parts |
| 18. | talc | 3.0 parts |
| 19. | iron oxide yellow | 0.9 part |
| 20. | iron oxide red | 0.3 part |
| 21. | iron oxide black | 0.1 part |

Note
*6): Cetyl PEG/PPG-10/1 dimethicone (long-chain alkyl · polyether co-modified silicone).
Note
*7): An amorphous ultrafinely divided titanium oxide composite produced according to the production method described in Example 2 of JP 06-199635 A.

### Production Method

Components 14 to 21 (powder components) are thoroughly mixed with a Super mixer. To this is added a solution prepared by dissolving components 10 to 13 (aqueous phase components). Mix into this a mixture of components 1 to 9 (oil phase components) that has been prepared in advance by mixing at 70° C. and then cool to room temperature.

### Effects

A high perception of transparency and a low pallidity are obtained during use. The ultraviolet protective performance is also excellent.

### Formulation Example 9

### Sunscreen

### Components

| | | |
|---|---|---|
| 1. | dimethylpolysiloxane (6 mm²/s) | 2.0 parts |
| 2. | product number: FA 4002 ID *8) | 1.0 part |
| 3. | dimethylpolysiloxane (2 mm²/s) | 18.2 parts |
| 4. | product number: SS-2910 *9) | 2.0 parts |
| 5. | purified water | 40.0 parts |
| 6. | preservative | 0.1 part |
| 7. | ethanol | 8.0 parts |
| 8. | alumina · silica-treated finely divided | 1.5 parts |
| | titanium oxide | |
| 9. | silicone-treated, iron-doped finely | 0.8 part |
| | divided titanium oxide | |
| 10. | octylalkoxysilane-treated finely | 3.2 parts |
| | divided titanium oxide | |
| 11. | spherical silicone powder with an | 2.0 parts |
| | average primary particle size of 3 μm | |
| 12. | spherical methacrylic resin beads with an | |
| | average primary particle size of 1 μm | 1.2 parts |
| 13. | W/O cream of Example 46 | 20.0 parts |

Note
*8): Isododecane solution of acrylates/polytrimethylsiloxymethacrylate copolymer, containing 40 weight % effective component.
Note
*9): PEG-10 dimethicone (polyether-modified silicone).

### Production Method

Mix components 1 to 4 (oil phase components) and disperse to uniformity; then add components 8 to 12 (powder components) and mill with a paint shaker. Then add a preliminarily prepared solution of components 5 to 7 (aqueous phase components) and mill further with a paint shaker. Fill the resulting dispersion and component 13 and stainless steel beads into a plastic bottle and stir and mix by shaking.

### Effects

A dry feel is obtained during use, but without a squeaky sensation. A high perception of transparency and a low pallidity are obtained. Resistant to unwanted removal of the cosmetic; the resistance to water and sebum and the ultraviolet protective performance are also excellent.

1. A cosmetic raw material comprising:
(A) a polyether-modified organopolysiloxane represented by general formula (1) below;
(B) a chain-form silicone oil that is a liquid at 25° C., does not contain a cyclic structure, and does not contain a resinous structure; and
(C) an oil that is a liquid at 30° C. and that satisfies the following conditions (c1) to (c4):
(c1) at least one hydroxyl group is present in each molecule,
(c2) from 0 to 3, as the number of moles of addition, oxyethylene units are present in each molecule,
(c3) an HLB value in the range from 0.1 to 6.0, and
(c4) an average molecular weight in the range from 200 to 7000, 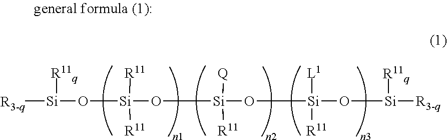
wherein
each R¹¹ is independently a substituted or unsubstituted C₁₋₃₀ monovalent hydrocarbyl group, a C₁₋₃₀ alkoxy group, a hydroxyl group, or a hydrogen atom,
L¹ is a siloxane dendron structure-containing silylalkyl group Lⁱ represented by general formula (2-1) when i=1, or is a chain-form organosiloxane group represented by general formula (2-2) or (2-3), 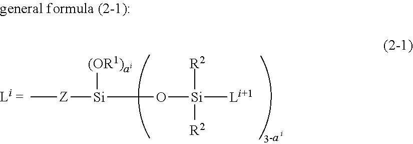
wherein each R¹ is independently a C₁₋₁₀ alkyl group or an aryl group, each R² is independently a C₁₋₆ alkyl group or a phenyl group, Z is a divalent organic group, i indicates the generation of the silylalkyl group represented by Lⁱ and is an integer from 1 to c, where c is the number of generations, which is the number of iterations of the indicated silylalkyl group, the number of generations c is an integer from 1 to 10, Lⁱ⁺¹ is the indicated silylalkyl group when i is less than c and is the methyl group or phenyl group when i=c, and aⁱ is a number in the range from 0 to 3, 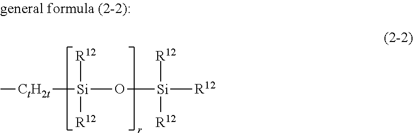
wherein each R¹² is independently a C₁₋₃₀ monovalent hydrocarbyl group, a hydroxyl group, or a hydrogen atom, t is a number in the range from 2 to 10, and r is a number in the range from 1 to 100, 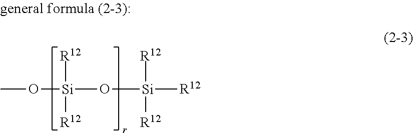
wherein R¹² is a group as defined above and r is a number in the range from 1 to 100,
Q is a polyether-modified group bonded to a silicon atom across an at least divalent linker group and contains an oxyalkylene unit represented by general formula (3-1) wherein at least 3 of the total oxyalkylene units are the oxyethylene unit,
general formula (3-1):
—CᵣH₂ᵣ—O— (3-1)
wherein r is a number in the range from 1 to 6,
R is a group selected from L¹, Q, and R¹¹,
n1, n2, and n3 are numbers in the ranges 4≦n1≦1000, 0≦n2≦50, and 0≦n3≦50,
q is an integer in the range from 0 to 3, and
when n2=0, q is an integer in the range from 1 to 3 and at least one R is Q.

2. The cosmetic raw material according to claim 1, wherein Q in general formula (1) is a polyether-modified group represented by general formula (4-1),
general formula (4-1):
—R³(—O—X¹ₘ—R⁴)ₚ (4-1)
wherein R³ is a (p+1)-valent organic group, p is an integer from 1 to 3, each X¹ is independently an oxyalkylene unit with the general formula (3-1) wherein at least three of the total oxyalkylene units represented by X¹ₘ are the oxyethylene unit, m is a number in the range from 3 to 100, and R⁴ is a group selected from the group consisting of a hydrogen atom, C₁₋₂₀ alkyl groups, and acyl groups.

3. The cosmetic raw material according to claim 1, wherein Q in general formula (1) is a polyether-modified group containing a polyoxyalkylene unit represented by general formula (3-1-1),
general formula (3-1-1):
—(C₂H₄O)ₜ₁(C₃H₆O)ₜ₂— (3-1-1)
wherein t1 is a number greater than 3, t2 is a number greater than or equal to 0, and (t1+t2) is a number in the range from 4 to 100.

4. The cosmetic raw material according to claim 1, wherein L¹ in general formula (1) is a silylalkyl group represented by general formula (2-1-1) or (2-1-2) below and when n3=0, q is an integer in the range from 1 to 3 and at least one of R is L¹, 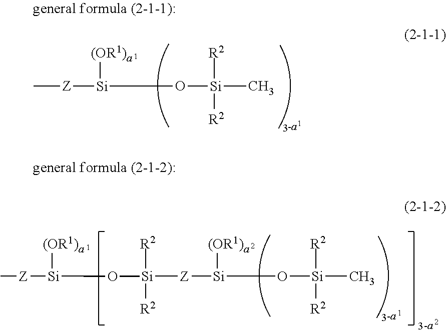
wherein R¹, R², and Z are groups as defined above and a¹ and a² are each independently a number in the range from 0 to 3.

5. The cosmetic raw material according to claim 1, wherein the HLB of component (A) is in the range from 0.1 to 6.0.

6. The cosmetic raw material according to claim 1, wherein component (B) is a chain-form silicone oil selected from (B-1) chain-form dimethylpolysiloxanes having a kinematic viscosity at 25° C. of not more than 20 mm²/s or (B-2) chain-form alkyl-modified methylpolysiloxanes having a kinematic viscosity at 25° C. of not more than 20 mm²/s.

7. The cosmetic raw material according to claim 1, wherein component (C) is a nonionic surfactant or a nonionic cosurfactant.

8. The cosmetic raw material according to claim 1, wherein component (C) is at least one oil selected from (C-1) higher alcohols, (C-2) fatty acid esters, (C-3) ethers, and (C-4) silicones having at least one hydroxyl group in the molecule excluding silicones that correspond to component (A) or component (B).

9. The cosmetic raw material according to claim 8, wherein component (C) is (C-1) a higher alcohol, (C-2) a fatty acid ester, or (C-3) an ether and has in its hydrophobic moiety a C₁₀₋₃₀ monovalent hydrocarbyl group.

10. The cosmetic raw material according to claim 8, wherein component (C) is (C-2) a fatty acid ester or (C-3) an ether and is a derivative obtained by the esterification or etherification of a polyhydric alcohol selected from sorbitan, sucrose, glycerol, polyglycerols, propylene glycol, and polypropylene glycols.

11. The cosmetic raw material according to claim 8, wherein component (C) is at least one silicone selected from alcohol-modified silicones, silanol-modified silicones, and phenol-modified silicones and has a kinematic viscosity at 25° C. of not more than 200 mm²/s.

12. The cosmetic raw material according to claim 1, having an appearance at 25° C. of a semi-transparent to transparent liquid.

13. The cosmetic raw material according to claim 1, wherein Q in general formula (1) is a polyether-modified group containing a polyoxyalkylene unit represented by general formula (3-1-1),
general formula (3-1-1):
—(C₂H₄O)ₜ₁(C₃H₆O)ₜ₂— (3-1-1)
wherein t1 is a number greater than 3, t2 is a number greater than or equal to 0, and (t1+t2) is a number in the range from 8 to 50.

14. The cosmetic raw material according to claim 2, wherein Q in general formula (1) is a polyether-modified group containing a polyoxyalkylene unit represented by general formula (3-1-1),
general formula (3-1-1):
—(C₂H₄O)ₜ₁(C₃H₆O)ₜ₂— (3-1-1)
wherein t1 is a number greater than 3, t2 is a number greater than or equal to 0, and (t1+t2) is a number in the range from 8 to 50.
